(19) United States
(12) Patent Application Publication
(10) Pub. No.: US 2007/0186296 A1
(43) Pub. Date: Aug. 9, 2007

(54) CLONING, CHARACTERIZATION, AND APPLICATION OF TNFRSF19 IN NEUROLOGICAL DISORDERS

(75) Inventors: Ying Gao, East Brunswick, NJ (US); Huai-Ping Ling, Princeton Junction, NJ (US); Andrew Wood, Newtown, PA (US); Kodangattil R. Sreekumar, Plainsboro, NJ (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/670,705

(22) Filed: Feb. 2, 2007

Related U.S. Application Data
(60) Provisional application No. 60/764,350, filed on Feb. 2, 2006.

(51) Int. Cl. A01K 67/027; C12Q 1/68; G01N 33/53; C07H 21/04; C12P 21/06; C12N 5/06; C07K 14/715

(52) U.S. Cl. 800/14; 435/6; 435/7.1; 435/69.1; 435/320.1; 435/343; 530/350; 536/23.5; 530/388.22

(57) ABSTRACT

TNFRSF19 nucleic acids and proteins, TNFRSF19 binding partners and modulators, and methods of using the same.

### RELATED APPLICATIONS

Priority is claimed to U.S. Provisional Patent Application No. 60/764,350, filed Feb. 2, 2006, which is incorporated herein in its entirety.

### FIELD OF THE INVENTION

The present invention generally relates to methods and compositions for nerve regeneration and other uses. More particularly, the present invention relates to TNFRSF19 nucleic acids and proteins, binding partners and modulators thereof, and the use of the same for applications such as developing preclinical models of and therapies for degenerative neurological disorders.

### BACKGROUND OF THE INVENTION

Inhibition of neurite outgrowth is a major obstacle for successful axon regeneration in the injured, diseased, and aging mammalian central nervous system (CNS). The lack of axonal growth in the CNS is based upon several factors, including formation of a glial scar, the absence of neurotrophic factors, the intrinsic growth state of the neurons, and the presence of growth-inhibitory molecules associated with myelin (MAIFs).

Three MAIFs have been identified, including myelin-associated glycoprotein (MAG), oligodendrocyte-myelin glycoprotein (OMgp), and Nogo-A (which includes two inhibitory subunits, Nogo-66 and amino-Nogo). All three MAIFs bind to a multi-protein complex that includes the ligand-binding Nogo-66 receptor (NgR1), a glycosylphosphatidyl-inositol (GPI-linked) protein, and two signal-transducing partners, the p75 neurotrophin receptor and the LINGO-1 protein. Binding of the MAIFs to this complex activates the small GTPase RhoA and leads to growth cone collapse. A dominant negative NgR can substantially reduce the inhibitory effects of MAG, Nogo-66, and OMpg (Wang et al. (2002) Nature 82:1566-1569; Domeniconi et al. (2002) Neuron 35:283). Similarly, knockdown of NgR, p75, and RhoA using single-stranded RNA interference (siRNA) was shown to disinhibit neurite outgrowth of dorsal root ganglia neurons (Ahmed et al. (2005) Mol. Cell. Neurosci. 28(3):509-523). Collectively, these results suggest that the NgR complex may be a useful target for therapeutic intervention following CNS injury. See generally, Schwab (2004) Curr. Opin. Neurobiol. 14:118-124; Kastin et al. (2005) Curr. Pharm. Design 11:1247-1253; and Mandemakers et al. (2005) Curr. Biol. 15(8): R302-205.

The role of p75 as an in vivo mediator of NgR and myelin-induced inhibition has been questioned, however, based upon the limited expression of p75 in the adult CNS. In particular, some neuron types that are inhibited by myelin, such as retinal ganglion cells, do not express p75 (Chao et al. (2003) Nat. Rev. Neurosci. 4:299-309). Further, significant myelin inhibitory activity remains in p75-deficient mice (Song et al. (2004) J.Neurosci. 24:542-546).

In contrast to p75, the tumor necrosis factor receptor family member TNFRSF19 (alternatively called TAJ, TRADE, or TROY) is broadly expressed in both developing and adult neurons in mouse and human (Hisaoka et al. (2000) Dev. Brain Res. 143:105-109; Pispa et al. (2003) Gene Expr. Patterns 3(5):675-67; Hisaoka et al. (2004) Glia 45:313-324). In human brain, expression is detected in most brain regions, including the amygdala, caudate, corpus callosum, hippocampus, thalamus, cerebellum, cortex, medulla, spinal cord, occipital lobe, frontal lobe, temporal lobe, and putamen. TNFRSF19 expression is also detected in purified cultures of CG (cerebellar granule) and DRG (dorsal root ganglion) neurons, as well as in non-neuronal cells, such as oligodendrocytes and astrocytes (Shao et al. (2005) Neuron 45(3):353-359). Based upon its expression profile and homology to p75, TNFRSF19 was suggested as a co-receptor for NgR in neurons.

Recent studies have shown that the TNFRSF19 may replace p75 in the NgR complex and participate as a functional MAIF receptor in vivo. Specifically, human TNFRSF19 is detected in a complex that includes NgR and LINGO-1 (a leucine-rich repeat (LRR) family member) and that activates RhoA. In addition, soluble human TNFRSF19 Fc/AP fusion proteins reverse neurite outgrowth inhibition of Cg neurons and DRG neurons caused by myelin inhibitors. Elevated neurite outgrowth (i.e., neurites with longer processes) is observed in MAIF-treated TNFRSF19-deficient murine neurons. See Shao et al. (2005) Neuron 45(3):353-359 and Park et al. (2005) Neuron 45(3):345-351.

Notwithstanding substantial interest in the NgR complex as a modulator of neurite outgrowth during axon regeneration, therapies that employ inhibitors of NgR are not yet available. The present invention provides isolated TNFRSF19 nucleic acids and proteins, methods for identifying TNFRSF19 inhibitors and other inhibitors of a functional NgR complex, and methods for using the same to promote neurite outgrowth.

### SUMMARY OF THE INVENTION

The present invention provides isolated TNFRSF19 nucleic acids and proteins, methods for identifying binding partners and modulators thereof, and methods for using the same.

Representative TNFRSF19 nucleic acids include (a) an open reading frame encoding a TNFRSF19 protein comprising an amino acid sequence at least 91% identical to SEQ ID NO:2 or 4; (b) an open reading frame encoding a TNFRSF19 protein comprising an amino acid sequence of SEQ ID NO:2 or 4; (c) a nucleotide sequence at least 93% identical to SEQ ID NO: 1 or 3; (d) a nucleotide sequence of SEQ ID NO:1 or 3; (e) a nucleic acid that specifically hybridizes to the complement of SEQ ID NO:1 or 3 under stringent hybridization conditions; or (f) a nucleotide sequence that is the complement of any one of (a)-(e). Also provided are vectors that include the disclosed TNFRSF19 nucleic acids and host cells expressing such vectors.

Representative TNFRSF19 proteins of the invention include proteins comprising (a) an amino acid sequence at least 91% identical to SEQ ID NO:2 or 4; or (b) an amino acid sequence of SEQ ID NO:2 or 4.

Also provided are antibodies and antibody fragments which specifically bind to the disclosed TNFRSF19 proteins, and which disinhibit neurite outgrowth.

Further provided is a genetically modified rat comprising a mutation in a nucleotide sequence encoding a TNFSRF19 protein of the invention, wherein the mutation alters (i) expression of a TNFRSF19 protein, or (ii) function of a TNFRSF19 protein in forming a receptor complex comprising TNFRSF19 and NgR.

The TNFRSF19 proteins of the invention may form a receptor complex comprising NgR, including NgR1, NgR2, or NgR3, and/or a receptor complex that blocks neurite outgrowth. Variant forms of a TNFRSF19 protein, for example, a TNFRSF19 protein comprising a dominant negative mutation, may disinhibit neurite outgrowth.

Further provided are methods for identifying binding partners and modulators of a TNFRSF19 protein. For example, a method for identifying TNFRSF19 binding partners may comprise the steps of: (a) providing a TNFRSF19 protein; (b) providing a one or more test agents to the TNFRSF19 protein under conditions sufficient for binding; (c) assaying binding of a test agent to the TNFRSF19 protein; and (d) selecting a test agent that demonstrates specific binding to the TNFRSF19 protein. A method for identifying TNFRSF19 modulators may comprise the steps of: (a) providing a TNFRSF19 protein under conditions for formation of a receptor complex comprising NgR; (b) providing one or more test agents or a control agent to the TNFRSF19 protein and NgR of (a); (c) allowing sufficient time for formation of a receptor complex; (d) assaying (i) formation of a receptor complex, (ii) capacity of the receptor complex of (c) to activate RhoA; or (iii) capacity of the receptor complex of (c) to inhibit neurite outgrowth; and (e) selecting a test agent that shows (i) altered formation of a receptor complex in the presence of the agent as compared to a control agent, (ii) altered capacity of the receptor complex of (c) to activate RhoA when in the presence of the test agent as compared to a control agent; or (iii) altered capacity of the receptor complex of (c) to inhibit neurite outgrowth when in the presence of the test agent as compared to a control agent. Test agents useful in these methods include peptides, proteins, oligomers, nucleic acids, small molecules, or antibodies.

Still further, the present invention provides modulators identified by the foregoing methods and methods for promoting neurite outgrowth in vitro or in vivo by contacting neurons with a TNFRSF19 binding partner or modulator identified by performing such methods.

### BRIEF DESCRIPTION OF THE DRAWINGS

FIGS. 1A-1C set forth the nucleotide and amino acid sequences of a virtual rat TNFRSF19 cDNA.

FIGS. 1A-1B illustrate SEQ ID NO:5, the 5′ untranslated region of a virtual rat TNFRSF19 cDNA; SEQ ID NO:1, the coding region of the virtual rat TNFRSF19 cDNA; and SEQ ID NO:6 the 3′ untranslated region of the virtual rat TNFRSF19 cDNA.

FIG. 1C illustrates SEQ ID NO:2, the translated sequence of SEQ ID NO:1. A signal peptide, two tumor necrosis factor receptor (TNFR) domains, and a transmembrane segment are indicated.

FIGS. 2A-2B set forth the nucleotide (SEQ ID NO:3) and amino acid (SEQ ID NO:4) sequences of rat TNFRSF19 cDNA isolated from rat brain. A signal peptide, two tumor necrosis factor receptor (TNFR) domains, and a transmembrane segment are indicated.

FIGS. 3A-3B show an alignment of the virtual rat TNFRSF19 cDNA open reading frame (SEQ ID NO:1) and rat TNFRSF19 isolated from rat brain (SEQ ID NO:3).

FIG. 3C shows an alignment of the translated rat TNFRSF19 virtual cDNA (SEQ ID NO:2) and the translated rat TNFRSF19 isolated from rat brain (SEQ ID NO:4).

FIGS. 4A-4C show the results of BLASTn analysis using the rat TNFRSF19 nucleotide sequence (SEQ ID NO:3) as a query sequence.

FIGS. 4D-4E show the results of BLASTP analysis using the rat TNFRSF19 amino acid sequence (SEQ ID NO:4) as a query sequence.

FIGS. 5A-5B depict Western blots prepared using 20 μg of CHO-K1 cell lysate per lane and analyzed using an anti-Myc antibody (FIG. 5A) or an anti-mouse TNFRSF19 antibody (FIG. 5B). Lane 1, cells transfected with empty vector (i.e., lacking a TNFRSF19 cDNA); lane 2, cells transfected with murine TNFRSF19; lane 3, cells transfected with rat TNFRSF19 with a Myc tag; arrow, position of the approximately 50 kDa TNFRSF19 protein.

FIG. 6 is a genomic map of the rat TNFRSF19 locus.

### DETAILED DESCRIPTION OF THE INVENTION

TNFRSF19 is a member of the tumor necrosis family receptor (TNFR) superfamily having a type I transmembrane domain and two complete and one partial cysteine-rich motifs in the extracellular region. Both murine and human cDNAs have been cloned (Hu et al., Genomics, 1999, 62(1):103-107; Kojima et al., Long et al, Scand. J. Immunol., 2000, 51(suppl. 1), 64). TNFRSF19 is capable of activating key signaling pathways of the TNF receptor family, such as Jnk and NFKb, and is also a co-receptor for Nogo-66 receptor (NgR1).

The present invention provides rat TNFRSF19 nucleic acids and proteins, variants thereof, and inhibitors thereof. Rat is one of the most commonly used species for in vitro cell-based assays and in vivo preclinical studies. Accordingly, the disclosed TNFRSF19 compositions are useful in such assays and preclinical studies pertaining to axon regeneration and cell death.

I. TNFRSF19 Nucleic Acids and Proteins

The present invention provides novel TNFRSF19 nucleic acids and proteins, including TNFRSF19 proteins that participate in a functional NgR complex. A functional NgR complex may include Nogo-66 receptor (NgR1), NgR2, or NgR3. See e.g., GenBank Accession Nos. Q9BZR6, AAM46772, AAK20166, AAP82838, NP_{—}848665, NP_{—}852045, AAP74960, AAP21838 . See also Oertle et al. (2003) J. Neurosci. 23 (13):5393-5406; Lauren et al. (2003) Mol. Cell. Neurosci. 24 (3):581-594; Pignot et al. (2003) J. Neurochem. 85 (3):717-728; Barton et al. (2003) EMBO J. 22 (13), 3291-3302; each of which is incorporated in its entirety herein. Representative TNFRSF19 nucleic acids of the invention are set forth as SEQ ID NOs:1 and 3, which encode the TNFRSF19 proteins of SEQ ID NOs:2 and 4, respectively.

I.A. TNFRSF19 Nucleic Acids

Nucleic acids are deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms nucleic acid molecule or nucleic acid may also be used in place of gene, cDNA, mRNA, or cRNA. Nucleic acids may be synthesized, or may be derived from any biological source, including any organism. Representative methods for cloning nucleic acids that encode a TNFRSF19 protein are described in Example 1.

Representative nucleic acids of the invention comprise the nucleotide sequence of SEQ ID NO:1 or 3 and substantially identical sequences, for example, sequences at least 93% identical to SEQ ID NO:1 or 3; such as at least 94% identical; or at least 95% identical; or at least 96% identical; or at least 97% identical; or at least 98% identical; or at least 99% identical. Sequences are compared for maximum correspondence using a sequence comparison algorithm using the full-length sequence of SEQ ID NO:1 or 3 as the query sequence, as described herein below, or by visual inspection. See also Example 1 and Table 1.

Substantially identical sequences may be polymorphic sequences, i.e, alternative sequences or alleles in a population. An allelic difference may be as small as one base pair. For example, SEQ ID NOs:1 and 3 are polymorphic sequences.

Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to the full length of SEQ ID NO:1 or 3 under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

A preferred nucleotide sequence employed for hybridization studies or assays includes probe sequences that are complementary to at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of SEQ ID NO:1 or 3. Such fragments may be readily prepared, for example by chemical synthesis of the fragment, by application of nucleic acid amplification technology, or by introducing selected sequences into recombinant vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under stringent conditions a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× SSC at 65° C. An example of stringent wash conditions is 15 minutes in 0.2× SSC buffer at 65° C. See Sambrook et al., eds (1989) Molecular Cloning: A Laboratory Manual, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1× SSC at 45° C. An example of low stringency wash for a duplex of more than about 100 nucleotides, is 15 minutes in 4× to 6× SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2× SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1× SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5× SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1× SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1× SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

The term conservatively substituted variants refers to nucleic acid sequences having degenerate codon substitutions wherein the third position of one or more selected (or all) codons is substituted with mixed-base and/or deoxyinosine residues. See Batzer et al. (1991) Nucleic Acids Res. 19:5081; Ohtsuka et al. (1985) J. Biol. Chem. 260:2605-2608; and Rossolini et al. (1994) Mol. Cell Probes 8:91-98.

Nucleic acids of the invention also comprise nucleic acids complementary to SEQ ID NO:1 or 3, and subsequences and elongated sequences of SEQ ID NO:1 or 3 and complementary sequences thereof. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The term subsequence refers to a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term elongated sequence refers to an addition of nucleotides (or other analogous molecules) incorporated into the nucleic acid. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, intronic sequences, additional restriction enzyme sites, multiple cloning sites, and other coding segments. Thus, the invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/New York; Ausubel (ed.) (1995) Short Protocols in Molecular Biology, 3rd ed. Wiley, New York.

In another aspect of the invention, a method is provided for detecting a nucleic acid molecule that encodes a TNFRSF19 protein. Such methods may be used to detect TNFRSF19 gene variants or altered gene expression. Sequences detected by methods of the invention may detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a TNFRSF19 nucleic acid molecule may be measured, for example, using an RT-PCR assay. See Chiang (1998) J. Chromatogr. A. 806:209-218, and references cited therein.

In another aspect of the invention, genetic assays based on TNFSRF19 nucleic acids may be used for quantitative trait loci (QTL) analysis and to screen for genetic variants, for example by allele-specific oligonucleotide (ASO) probe analysis (Conner et al., 1983), oligonucleotide ligation assays (OLAs) (Nickerson et al., 1990), single-strand conformation polymorphism (SSCP) analysis (Orita et al., 1989), SSCP/heteroduplex analysis, enzyme mismatch cleavage, direct sequence analysis of amplified exons (Kestila et al., 1998; Yuan et al., 1999), allele-specific hybridization (Stoneking et al., 1991), and restriction analysis of amplified genomic DNA containing the specific mutation. Automated methods may also be applied to large-scale characterization of single nucleotide polymorphisms (Wang et al., 1998; Brookes, 1999). Preferred detection methods are non-electrophoretic, including, for example, the TAQMAN™ allelic discrimination assay, PCR-OLA, molecular beacons, padlock probes, and well fluorescence. See Landegren et al. (1998) Genome Res 8:769-776 and references cited therein.

I.B. TNFRSF19 Proteins

The present invention also provides isolated TNFRSF19 polypeptides. Polypeptides and proteins each refer to a compound made up of a single chain of amino acids joined by peptide bonds. Representative TNFRSF19 polypeptides are set forth as SEQ ID NOs:2 and 4. Additional polypeptides of the invention include polypeptides having an amino acid sequence that is at least 91% identical to SEQ ID NO:2 or 4, for example, at least 92% identical, or at least 93% identical, or at least 94% identical, or at least 95% identical, or at least 96% identical, or at least 97% identical, or at least 98% identical, or at least 99% identical. Sequences are compared for maximum correspondence using a sequence comparison algorithm using the full-length sequence of SEQ ID NO:2 or 4 as the query sequence, as described herein below, or by visual inspection. See also Example 1 and Table 1. The invention further encompasses polypeptides encoded by any one of the nucleic acids disclosed herein.

Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; 0-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

The present invention also provides functional fragments of a TNFRSF19 polypeptide, for example, fragments that binds to Nogo-66 receptor (NgR1) and/or LINGO-1. Functional polypeptide sequences that are longer than the disclosed sequences are also provided. For example, one or more amino acids may be added to the N-terminus or C-terminus of an antibody polypeptide. Such additional amino acids may be employed in a variety of applications, including but not limited to purification applications. Methods of preparing elongated proteins are known in the art.

TNFSRF19 proteins of the invention include proteins comprising amino acids that are conservatively substituted variants of SEQ ID NO:2 or 4. A conservatively substituted variant refers to a polypeptide comprising an amino acid in which one or more residues have been conservatively substituted with a functionally similar residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Isolated polypeptides of the invention may be purified and characterized using a variety of standard techniques that are known to the skilled artisan. See e.g., Schröder & Lübke (1965) The Peptides. Academic Press, New York; Bodanszky (1993) Principles of Peptide Synthesis, 2nd rev. ed. Springer-Verlag, Berlin/New York; Ausubel (ed.) (1995) Short Protocols in Molecular Biology, 3rd ed. Wiley, N.Y.

The present invention further provides methods for detecting a TNFRSF19 polypeptide. The disclosed methods can be used, for example, to determine altered levels of TNFRSF19 protein, for example, induced levels of TNFRSF19 protein, or to detect TNFRSF19 in a myelin inhibitory complex.

For example, the method may involve performing an immunochemical reaction with an antibody that specifically recognizes a TNFRSF19 protein. Techniques for detecting such antibody-antigen conjugates or complexes are known in the art and include but are not limited to centrifugation, affinity chromatography and other immunochemical methods. See e.g., Ishikawa (1999) Ultrasensitive and Rapid Enzyme Immunoassay. Elsevier, Amsterdam/New York, United States of America; Law (1996) Immunoassay: A Practical Guide. Taylor & Francis, London/Bristol, Pa., United States of America; Liddell & Weeks (1995) Antibody Technology. Bios Scientific Publishers, Oxford, United Kingdom; and references cited therein.

I.C. Nucleotide and Amino Acid Sequence Comparisons

The terms identical or percent identity in the context of two or more nucleotide or protein sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term substantially identical in regards to a nucleotide or protein sequence means that a particular sequence varies from the sequence of a naturally occurring sequence by one or more deletions, substitutions, or additions, the net effect of which is to retain biological function of a TNFRSF19 nucleic acid or protein.

For comparison of two or more sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison may be conducted, for example, by the local homology algorithm of Smith & Waterman (1981) Adv. Appl. Math 2:482-489, by the homology alignment algorithm of Needleman & Wunsch (1970) J. Mol. Biol. 48:443-453, by the search for similarity method of Pearson & Lipman (1988) Proc. Natl. Acad. Sci. USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.) (1995) Short Protocols in Molecular Biology, 3rd ed. Wiley, N.Y.

A preferred algorithm for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (http://www.ncbi.nlm.nih.gov/). The BLAST algorithm parameters determine the sensitivity and speed of the alignment. For comparison of two nucleotide sequences, the BLASTn default parameters are set at W=11 (wordlength) and E=10 (expectation), and also include use of a low-complexity filter to mask residues of the query sequence having low compositional complexity. For comparison of two amino acid sequences, the BLASTp program default parameters are set at W=3 (wordlength), E=10 (expectation), use of the BLOSUM62 scoring matrix, gap costs of existence=11 and extension=1, and use of a low-complexity filter to mask residues of the query sequence having low compositional complexity. See Example 1.

II. System for Recombinant Expression of a TNFRSF19 Protein

The present invention further provides a system for expression of a recombinant TNFRSF19 protein. Such a system may be used for subsequent purification and/or characterization of a TNFRSF19 protein. For example, a purified TNFRSF19 protein may be used as an immunogen for the production of an TNFRSF19 antibody, described further herein below. A system for recombinant expression of a TNFRSF19 protein may also be used for identification of inhibitors of a TNFRSF19 protein, as described further herein below.

An expression system refers to a host cell comprising a heterologous nucleic acid and the protein encoded by the heterologous nucleic acid. For example, a heterologous expression system may comprise a host cell transfected with a construct comprising a recombinant TNFRSF19 nucleic acid, a host cell transfected with TNFRSF19 cRNA, a host cell transfecting with a construct comprising a vector and a nucleic acid molecule encoding a TNFRSF19 protein operatively linked to a promoter, or a cell line produced by introduction of heterologous nucleic acids into a host cell genome. expressed. The system may further comprise one or more additional heterologous nucleic acids relevant to TNFRSF19 function, such as components of a Nogo-66 receptor (NgR1) receptor complex. See Example 1. These additional nucleic acids may be expressed as a single construct or multiple constructs.

Isolated proteins and recombinantly produced proteins may be purified and characterized using a variety of standard techniques that are known to the skilled artisan. See e.g., Schröder & Lübke (1965) The Peptides. Academic Press, New York; Schneider & Eberle (1993) Peptides, 1992: Proceedings of the Twenty-Second European Peptide Symposium. Sep. 13-19, 1992, Interlaken, Switzerland. Escom, Leiden; Bodanszky (1993) Principles of Peptide Synthesis, 2nd rev. ed. Springer-Verlag, Berlin/New York; Ausubel (ed.) (1995) Short Protocols in Molecular Biology, 3rd ed. Wiley, N.Y.

In one aspect of the invention, a recombinantly expressed TNFRSF19 protein is capable of interacting with Nogo-66 receptor (NgRi) and LINGO-1 proteins to form a functional Nogo-66 receptor (NgR1) complex. In another aspect, a recombinantly expressed TNFRSF19 protein is capable of interacting with Nogo-66 receptor (NgR1) and LINGO-1 to thereby disrupt function of the Nogo-66 receptor (NgR1) complex, i.e., a TNFRSF19 protein that has a dominant negative effect. Representative methods for determining functionality of a Nogo-66 receptor (NgR1) complex are described in Examples 2 and 3.

II.A. Expression Constructs

A construct for expression of a TNFRSF19 protein may include a vector and a TNFRSF19 nucleotide sequence, wherein the TNFRSF19 nucleotide sequence is operatively linked to a promoter sequence. A construct for recombinant TNFRSF19 expression may also comprise transcription termination signals and sequences required for proper translation of the nucleotide sequence. Preparation of an expression construct, including addition of translation and termination signal sequences, is known to one skilled in the art.

Recombinant production of a TNFRSF19 protein may be directed by a constitutive promoter or an inducible promoter. Representative promoters that may be used in accordance with the present invention include Simian virus 40 early promoter, a long terminal repeat promoter from retrovirus, an actin promoter, a heat shock promoter, and a metallothien protein.

Suitable vectors that may be used to express a TNFRSF19 protein include but are not limited to viruses such as vaccinia virus or adenovirus, baculovirus vectors, yeast vectors, bacteriophage vectors (e.g., lambda phage), plasmid and cosmid DNA vectors, transposon-mediated transformation vectors, and derivatives thereof.

Constructs are introduced into a host cell using a transfection method compatible with the vector employed. Standard transfection methods include electroporation, DEAE-Dextran transfection, calcium phosphate precipitation, liposome-mediated transfection, transposon-mediated transformation, infection using a retrovirus, particle-mediated gene transfer, hyper-velocity gene transfer, and combinations thereof.

II.B. Host Cells

Host cells are cells into which a heterologous nucleic acid molecule may be introduced. Representative host cells include eukaryotic hosts such as mammalian cells (e.g., HEK-293 cells, HeLa cells, CV-1 cells, COS cells), amphibian cells (e.g., Xenopus oocytes), insect cells (e.g., Sf9 cells), as well as prokaryotic hosts such as E. coli and Bacillus subtilis. Preferred host cells for functional assays substantially or completely lack endogenous expression of a TNFRSF19 protein.

A host cell strain may be chosen which modulates the expression of the recombinant sequence, or modifies and processes the gene product in a specific fashion desired. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

The present invention further encompasses recombinant expression of a TNFRSF19 protein in a stable cell line. Methods for generating a stable cell line following transformation of a heterologous construct into a host cell are known in the art. See e.g., Joyner (1993) Gene Targeting: A Practical Approach. Oxford University Press, Oxford/New York. Thus, transformed cells, tissues, or non-human organisms are understood to encompass not only the end product of a transformation process, but also transgenic progeny or propagated forms thereof.

Transiently transfected cells and cells of a stable cell line expressing TNFRSF19 may be frozen and stored for later use. Frozen cells may be readily transported for use at a remote location. Methods for preparation and handling of frozen cells may be found in Freshney (1987) Culture of Animal Cells: A Manual of Basic Technique, 2nd ed. A. R. Liss, New York and in U.S. Pat. Nos. 6,176,089; 6,140,123; 5,629,145; and 4,455,842; among other places.

III. Transgenic Animals

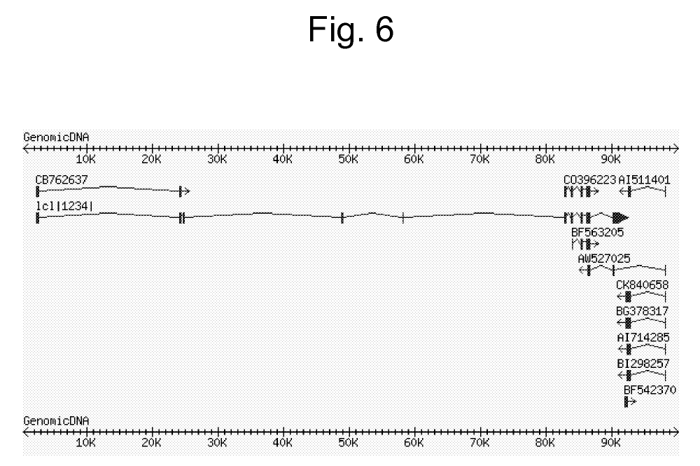

The present invention also provides a transgenic animal comprising a disruption of a TNFRSF19 locus. A disrupted gene may include expression of an altered level of TNFRSF19 or mutated variant of a TNFRSF19 gene. Rat strains with complete or partial functional inactivation of the TNFRSF19 gene in all somatic cells may be generated using standard techniques of site-specific recombination in embryonic stem cells. See Capecchi (1989) Science 244:1288-1292; Thomas & Capecchi (1990) Nature 346:847-850; and Delpire et al. (1999) Nat Genet 22:192-195. The present invention provides genomic mapping data useful for preparation of constructs targeted to the TNFRSF19 locus. See Example 4 and FIG. 6.

A transgenic animal in accordance with the present invention may also be prepared using anti-sense or ribozyme TNFRSF19 constructs, driven by a universal or tissue-specific promoter, to reduce levels of TNFRSF19 gene expression in somatic cells, thus achieving a “knock-down” phenotype. The present invention also provides the generation of rat strains with conditional or inducible inactivation of TNFRSF19. Such strains may also comprise additional synthetic or naturally occurring mutations, for example a mutation in Nogo-66 receptor (NgR1), LINGO-1, p75, other components of a Nogo-66 receptor (NgR1) complex, or upstream or downstream signaling components of a Nogo-66 receptor (NgR1) complex.

The present invention also provides transgenic animals with specific “knocked-in” modifications in the disclosed TNFRSF19 gene, for example to create an over-expression or dominant negative phenotype. Thus, “knocked-in” modifications include the expression of both wild type and mutated forms of a nucleic acid encoding a TNFRSF19 protein. Knock-in transgenic organisms may be made in any relevant species.

Techniques for the preparation of transgenic animals are known in the art. Exemplary techniques are described in U.S. Pat. No. 5,489,742 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species), the entire contents of each of which are herein incorporated by reference.

IV. TNFRSF19 Antibodies

In another aspect of the invention, a method is provided for producing an antibody that specifically binds a TNFRSF19 protein. According to the method, a full-length recombinant TNFRSF19 protein is formulated so that it may be used as an effective immunogen, and used to immunize an animal so as to generate an immune response in the animal. The immune response is characterized by the production of antibodies that may be collected from the blood serum of the animal. The present invention also provides antibodies produced by methods that employ the novel TNFRSF19 proteins disclosed herein, including SEQ ID NOs:2 and 4.

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Humanized antibodies of the invention include diabodies, tetrameric antibodies, single chain antibodies, tretravalent antibodies, multispecific antibodies (e.g., bispecific antibodies), and domain-specific antibodies that recognize a particular epitope. Cell lines that produce anti-TNFSRF19 antibodies are also encompassed by the invention.

Specific binding of an antibody to a TNFRSF19 protein refers to preferential binding to a TNFRSF19 protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

Chimeric antibodies comprise sequences from at least two different species. Humanized antibodies are one type of chimeric antibody.

The term humanized more specifically describes an antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. These general approaches may be combined with standard mutagenesis and synthesis techniques to produce an anti-TNFRSF19 antibody of any desired sequence.

The antibodies of this invention may also be human monoclonal antibodies, for example those produced by immortalized human cells, by SCID-hu mice or other non-human animals capable of producing human antibodies. Human antibodies may also be isolated from antibody phage libraries, for example, as described by Marks et al. (1991) J. Mol. Biol., 222:581-597. Chain shuffling and recombination techniques may be used to produce phage libraries having increased antibody diversity, e.g., libraries including antibodies with increased binding affinity. See Marks et al. (1992) Biotechnology 10:779-783 and Waterhouse et al. (1993) Nuc. Acids. Res. 21:2265-2266.

Antibodies having a tetrameric structure, similar to naturally occurring antibodies, may be recombinantly prepared using standard techniques. Recombinantly produced antibodies also include single chain antibodies, wherein the variable regions of a single light chain and heavy chain pair include an antigen binding region, and fusion proteins, wherein a variable region of an anti-TNFRSF19 antibody is fused to an effector sequence, such as an Fc domain, a cytokine, an immunostimulant, a cytotoxin, or any other therapeutic protein. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561.

Tetravalent antibodies (H₄L₄) comprising two intact tetrameric antibodies, including homodimers and heterodimers, may be prepared for example as described in PCT International Publication No. WO 02/096948. Antibody dimers may also be prepared via introduction of cysteine residue(s) in the antibody constant region, which promote interchain disulfide bond formation, using heterobifunctional cross-linkers (Wolff et al. (1993) Cancer Res. 53: 2560-5), or by recombinant production to include a dual constant region (Stevenson et al. (1989) Anticancer Drug Des. 3: 219-30).

TNFRSF19 antibodies prepared as disclosed herein may be used in methods known in the art relating to the localization and activity of TNFRSF19 proteins, e.g., for cloning of nucleic acids encoding a TNFRSF19 protein, immunopurification of a TNFRSF19 protein, imaging a TNFRSF19 protein in a biological sample, and measuring levels of a TNFRSF19 protein in appropriate biological samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art.

V. TNFRSF19 Binding Partners and Modulators

The present invention further discloses assays to identify binding partners and modulators of TNFRSF19 activity. Binding partners include proteins or other molecules that bind to TNFRSF19 and/or participate in a functional NgR complex with TNFRSF19 and NgR1, NgR2, or NgR3. TNFRSF19 modulators, i.e., agonists/activators and antagonists/inhibitors, are agents that alter chemical and biological activities or properties of a TNFRSF19 protein. Methods for identifying modulators involve assaying a reduced or enhanced level or quality of TNFRSF19 function in the presence of one or more test agents. Representative TNFRSF19 modulators include excess or variant TNFRSF19 proteins that mimic TNFRSF19 activity or that elicit a dominant negative phenotype, i.e., antagonism of TNFRSF19 function, and TNFRSF19 antibodies, as described herein above.

A control level or quality of TNFRSF19 activity refers to a level or quality of wild type TNFRSF19 activity, for example, when using a recombinant expression system comprising expression of SEQ ID NO:2 or 4. When evaluating the modulating capacity of a test agent, a control level or quality of TNFRSF19 activity comprises a level or quality of activity in the absence of the test agent.

Significantly changed activity of a TNFRSF19 protein refers to a quantifiable change in a measurable quality that is larger than the margin of error inherent in the measurement technique. For example, significant inhibition refers to TNFRSF19 activity that is reduced by about 2-fold or greater relative to a control measurement, or an about 5-fold or greater reduction, or an about 10-fold or greater reduction. Similarly, significant activation or agonism refers to significant inhibition refers to TNFRSF19 activity that is enhanced by about 2-fold or greater relative to a control measurement, or an about 5-fold or greater enhancement, or an about 10-fold or greater enhancement.

An assay of TNFRSF19 function may comprise determining a level of TNFRSF19 gene expression; determining binding activity of a recombinantly expressed TNFRSF19 protein; determining an active conformation of a TNFRSF19 protein; or determining activation of signaling events in response to binding of a TNFRSF19 modulator.

In accordance with the present invention there is also provided a rapid and high throughput screening method that relies on the methods described herein. This screening method comprises separately contacting a TNFRSF19 protein with a plurality of test agents. In such a screening method the plurality of target agents preferably comprises more than about 10⁴ samples, or more preferably comprises more than about 10⁵ samples, and still more preferably more than about 10⁶ samples.

The in vitro and cellular assays of the invention may comprise soluble assays, or may further comprise a solid phase substrate for immobilizing one or more components of the assay. For example, a TNFRSF19 protein, or a cell expressing a TNFRSF19 protein, may be bound directly to a solid state component via a covalent or non-covalent linkage. Optionally, the binding may include a linker molecule or tag that mediates indirect binding of a TNFRSF19 protein to a substrate.

V.A. Test Agents

A test agent refers to any agent that potentially interacts with TNFRSF19 protein, including any synthetic, recombinant, or natural product or composition. A test agent suspected to interact with a protein may be evaluated for such an interaction using the methods disclosed herein.

Representative test agents include but are not limited to peptides, proteins, nucleic acids (e.g., aptamers), small molecules (e.g., chemical compounds), antibodies or fragments thereof, nucleic acid-protein fusions, any other affinity agent, and combinations thereof. A test agent to be tested may be a purified molecule, a homogenous sample, or a mixture of molecules or compounds.

A small molecule refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Test agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of test agents in a library may be assayed simultaneously. Optionally, test agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. No. 6,180,348 and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667988), a library of nucleic acid-protein fusions (U.S. Pat. No. 6,214,553), and a library of any other affinity agent that may potentially bind to a TNFRSF19 protein (e.g., U.S. Pat. Nos. 5,948,635, 5,747,334, and 5,498,538).

A library may comprise a random collection of molecules. Alternatively, a library may comprise a collection of molecules having a bias for a particular sequence, structure, or conformation. See e.g., U.S. Pat. Nos. 5,264,563 and 5,824,483. Methods for preparing libraries containing diverse populations of various types of molecules are known in the art, for example as described in U.S. Patents cited herein above. Numerous libraries are also commercially available.

V.B. Binding Assays

In another aspect of the invention, a method for identifying of a TNFRSF19 modulator comprises determining specific binding of a test agent to a TNFRSF19 protein. For example, a method for identifying a TNFRSF19 binding partner may comprise: (a) providing a TNFRSF19 protein of claim 8; (b) providing a one or more test agents to the TNFRSF19 protein under conditions sufficient for binding; (c) assaying binding of a test agent to the isolated TNFRSF19 protein; and (d) selecting a test agent that demonstrates specific binding to the TNFRSF19 protein. Specific binding may also encompass a quality or state of mutual action such that binding of a test agent to a TNFRSF19 protein is modulatory. Alternatively, a binding partner as identified herein may be an endogenous component of a functional NgR complex that includes TNFRSF19.

Specific binding refers to a binding reaction which is determinative of the presence of the protein in a heterogeneous population of proteins and other biological materials. The binding of a test agent to a TNFRSF19 protein may be considered specific if the binding affinity is about 1×10⁴M⁻¹ to about 1×10⁶M⁻¹ or greater. Specific binding also refers to saturable binding. To demonstrate saturable binding of a test agent to a TNFRSF19 protein, Scatchard analysis may be carried out as described, for example, by Mak et al. (1989) J. Biol. Chem. 264:21613-21618.

Several techniques may be used to detect interactions between a TNFRSF19 protein and a test agent without employing a known competitive inhibitor. Representative methods include, but are not limited to, Fluorescence Correlation Spectroscopy, Surface-Enhanced Laser Desorption/lonization Time-Of-flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume (Tallgren, 1980). The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a TNFRSF19 protein) is expressed as a recombinant protein with a sequence tag, such as a poly-histidine sequence, inserted at the N-terminus or C-terminus. The expression is mediated in a host cell, such as E.coli, yeast, Xenopus oocytes, or mammalian cells. The protein is purified using chromatographic methods. For example, the poly-histidine tag may be used to bind the expressed protein to a metal chelate column such as Ni²+ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/lonization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., 1998). In a typical experiment, a target protein (e.g., a TNFRSF19 protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a TNFRSF19 protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al., 1983; Malmquist, 1993). In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al. (1999) Sensors and Actuators 54:3-15 and references therein.

V.C. Conformational Assay

The present invention also provides methods for identifying TNFRSF19 binding partners and modulators that rely on a conformational change of a TNFRSF19 protein when bound by or otherwise interacting with a test agent. For example, application of circular dichroism to solutions of macromolecules reveals the conformational states of these macromolecules. The technique may distinguish random coil, alpha helix, and beta chain conformational states.

To identify modulators of a TNFRSF19 protein, circular dichroism analysis may be performed using a recombinantly expressed TNFRSF19 protein. A TNFRSF19 protein is purified, for example by ion exchange and size exclusion chromatography, and mixed with a test agent. The mixture is subjected to circular dichroism. The conformation of a TNFRSF19 protein in the presence of a test agent is compared to a conformation of a TNFRSF19 protein in the absence of the test agent. A change in conformational state of a TNFRSF19 protein in the presence of a test agent identifies a TNFRSF19 binding partner or modulator. Representative methods are described in U.S. Pat. Nos. 5,776,859 and 5,780,242. Agonistic or antagonistic activity of the modulator may be assessed using functional assays, such as, the RhoA activation and neurite outgrowth assays described herein.

V.D. Functional Assays

In a preferred embodiment of the invention, a method for identifying a TNFRSF19 modulator employs a functional TNFRSF19 protein. Novel TNFRSF19 proteins disclosed herein include SEQ ID NOs:2 and 4. Representative methods for determining TNFRSF19 function include assaying any physiological change elicited by TNFRSF19 activity, including TNFRSF19 participation in activity of an NgR complex.

For example, a method for identifying a TNFRSF19 inhibitor may comprise (a) providing a TNFRSF19 protein under conditions for formation of a receptor complex comprising a NgR protein (e.g., Nogo-66 receptor (NgR1), NgR2, and NgR2) and LINGO-1 protein or agonist thereof; (b) providing one or more test agents or a control agent to the TNFRSF19 protein, NgR, and LINGO-1 protein of (a); (c) allowing sufficient time for formation of a receptor complex; (d) assaying (i) formation of a receptor complex, (ii) capacity of the receptor complex of (c) to activate RhoA; or (iii) capacity of the receptor complex of (c) to modulate neurite outgrowth; and (e) selecting a test agent that shows (i) altered formation of a receptor complex in the presence of the agent as compared to a control agent, (ii) altered capacity of the receptor complex of (c) to activate RhoA when in the presence of the test agent as compared to a control agent; or (iii) altered capacity of the receptor complex of (c) to modulate neurite outgrowth when in the presence of the test agent as compared to a control agent.

In accordance with the method, cells expressing TNFRSF19 may be provided in the form of a kit useful for performing an assay of TNFRSF19 function. Thus, cells may be frozen as described herein above and transported while frozen to others for performance of an assay. For example, a test kit for detecting a TNFRSF19 may include frozen cells transfected with DNA encoding a full-length TNFRSF19 protein and a medium for growing the cells.

Assays employing cells expressing recombinant TNFRSF19 may additionally employ control cells that are substantially devoid of native TNFRSF19 and proteins substantially similar to a TNFRSF19 protein. When using transiently transfected cells, a control cell may comprise, for example, an untransfected host cell. When using a stable cell line expressing a TNFRSF19 protein, a control cell may comprise, for example, a parent cell line used to derive the TNFRSF19-expressing cell line.

Assays of TNFRSF19 activity that employ transiently transfected cells may include a marker that distinguishes transfected cells from non-transfected cells. A marker may be encoded by or otherwise associated with a construct for TNFRSF19 expression, such that cells are simultaneously transfected with a nucleic acid molecule encoding TNFRSF19 and the marker. Representative detectable molecules that are useful as markers include but are not limited to a heterologous nucleic acid, a protein encoded by a transfected construct (e.g., an enzyme or a fluorescent protein), a binding protein, and an antigen.

V.E. Rational Design

The knowledge of the structure a native TNFRSF19 protein provides an approach for rational design of TNFRSF19 inhibitors. In brief, the structure of a TNFRSF19 protein may be determined by X-ray crystallography and/or by computational algorithms that generate three-dimensional representations. See Saqi et al. (1999) Bioinformatics 15:521-522; Huang et al. (2000) Pac Symp Biocomput:230-241; and PCT International Publication No. WO 99/26966. Alternatively, a working model of a TNFRSF19 protein structure may be derived by homology modeling (Maalouf et al., 1998). Computer models may further predict binding of a protein structure to various substrate molecules that may be synthesized and tested using the assays described herein above. Additional compound design techniques are described in U.S. Pat. Nos. 5,834,228 and 5,872,011.

In general, a TNFRSF19 protein is a membrane protein, and may be purified in soluble form using detergents or other suitable amphiphilic molecules. The resulting TNFRSF19 protein is in sufficient purity and concentration for crystallization. The purified TNFRSF19 protein may be crystallized under varying conditions of at least one of the following: pH, buffer type, buffer concentration, salt type, polymer type, polymer concentration, other precipitating ligands, and concentration of purified TNFRSF19. Methods for generating a crystalline protein are known in the art and may be reasonably adapted for determination of a TNFRSF19 protein as disclosed herein. See e.g., Deisenhofer et al. (1984) J. Mol. Biol. 180:385-398; Weiss et al. (1990) FEBS Lett. 267:268-272; or the methods provided in a commercial kit, such as the CRYSTAL SCREEN™ kit (available from Hampton Research of Riverside, Calif., United States of America).

A crystallized TNFRSF19 protein may be tested for functional activity and differently sized and shaped crystals are further tested for suitability in X-ray diffraction. Generally, larger crystals provide better crystallography than smaller crystals, and thicker crystals provide better crystallography than thinner crystals. Preferably, TNFRSF19 crystals range in size from 0.1-1.5 mm. These crystals diffract X-rays to at least 10 Å resolution, such as 1.5-10.0 Å or any range of value therein, such as 1.5, 1.6, 1.7,1.8,1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å or less being preferred for the highest resolution.

VI. Applications

The disclosed TNFRSF19 binding partners and TNFRSF19 modulators are useful both in vitro and in vivo for applications generally related to neuronal growth, survival, repair, regeneration, plasticity, etc.

In one aspect of the invention, TNFRSF19 inhibitors may be used to disinhibit neurite outgrowth or axon sprouting. Neurons that may be subject to TNFRSF19 inhibitors include injured neurons, neurons at risk of dying, or any other neuron for which an axon outgrowth response is desired, including neurons of the central nervous system or the peripheral nervous system.

For in vivo applications employing TNFRSF19 inhibitors, the subject may have suffered neuronal injury, such as a spinal cord injury, stroke, or closed-head injuries; or the subject may be suffering from or at risk of neuronal degeneration as a result of normal aging; or the subject may be a patient with a disease wherein neuron degeneration and resulting dysfunction plays a role in the pathophysiology. Relevant diseases include Alzheimer's disease (AD), Parkinson's disease, dementia with Lewy bodies, multisystem atrophy, frontotemporal dementia, Huntington's disease, corticobasal degeneration, spinocerebellar ataxias, supranuclear palsy, leukodystrophy, prion-related dementias, dementia resulting from brain tumors or cerebrovascular disorders, and multiple sclerosis. The subject may also be at risk for developing any one of the above-noted conditions, for example, subjects having autosomal dominant mutations that confer susceptibility to AD (mutations in β-amyloid precursor protein, presenilin 1, or presenilin 2) or the apolipoprotein E polymorphism associated with higher risk of developing AD.

In another aspect of the invention, TNFRSF19 proteins or agonists may be useful for inducing paraptosis-like cell death, i.e., a type of non-apoptotic cell death that occurs independently of oligonucleosomal DNA fragmentation and caspase activation. See Sperandio et al. (2000) Proc. Natl. Acad. Sci. USA 97: 14376-14381; Eby et al. (2000) J. Biol. Chem. 19;275(20):15336-15342; and Wang et al. (2004) J Cell Sci. 117:1525-1532. Recent studies implicate parapototic cell death in glioma cells (Chen et al. (2002) Blood 100:1371-1380; Mochizuki et al. (2002) J. Biol. Chem. 277:2790-2797). Neurons that may be subject to TNFRSF19 agonists or activators include cancerous neurons or other abnormal neurons for which activation of a cell death program is desirable. Conversely, TNFRSF19 inhibitors may be useful for inhibiting cell death of cells in which this alternative form of cell death is operative.

The present invention provides that an effective amount of a TNFRSF19 nucleic acid, a TNFRSF19 protein, or a TNFRSF19 modulator is administered to a subject, i.e., an amount sufficient to elicit a desired biological response. For example, an effective amount of a TNFRSF19 inhibitor may comprise an amount sufficient to elicit measurable neurite outgrowth, reduced RhoA activation in neurons treated with a TNFRSF19 inhibitor, and/or improvement of sensory and motor nerve functions. An effective amount of a TNFRSF19 protein may comprise an amount sufficient to induce paraptotic cell death.

For administration to a subject, TNFSRF19 modulators are formulated a pharmaceutically acceptable carrier, for example, large slowly metabolized macromolecules such as proteins, proteins, liposomes, polysaccharides, polylactic acids, polyglycolic acids, polymeric amino acids, amino acid copolymers, and inactive virus particles. Pharmaceutically acceptable salts may also be used, for example, mineral acid salts, such as hydrochlorides, hydrobromides, phosphates and sulfates, or salts of organic acids, such as acetates, propionates, malonates and benzoates. Formulations may additionally contain liquids such as water, saline, glycerol, and ethanol, and/or auxiliary agents, such as wetting or emulsifying agents or pH buffering agents, may be present in such compositions.

TNFRSF19 modulator s are administered in any matter suitable for contacting neurons subject to growth inhibition. A variety of techniques are available for promoting transfer of therapeutic agents across the blood brain barrier including disruption by surgery or injection, drugs which transiently open adhesion contact between CNS vasculature endothelial cells, and compounds which facilitate translocation through such cells. The compositions may also be administered by intrathecal or intraventricular injection, infusion, intraocular administration, or within/on implants, e.g., matrices such as collagen fibers or protein polymers, via cell bombardment, in osmotic pumps, grafts comprising appropriately transformed cells, etc. The administration of TNFRSF19 modulators may be localized so as to promote directional neurite outgrowth and axon regeneration.

Administration regimens may include a single injection or multiple injections. The selected dosage level and regimen will depend upon a variety of factors including the activity and stability (i.e., half life) of the therapeutic composition, formulation, the route of administration, combination with other drugs or treatments, the disease or disorder to be detected and/or treated, and the physical condition and prior medical history of the subject being treated.

TNFRSF19 modulators of the invention may be administered as an initial treatment, or for treatment of conditions that are unresponsive to conventional therapies. In addition, the TNFRSF19 modulators may be used in combination with other therapies to thereby elicit additive or potentiated therapeutic effects. TNFRSF19 modulators may be co-administered or co-formulated with additional agents, or formulated for consecutive administration in either order.

Representative agents useful for combination therapy include agents that similarly participate in disinhibition of neurite outgrowth by disruption of NgR complex signaling (e.g., inhibitors of NgR1, NgR2, NgR3, LINGO-1, or p75) and modulators that increase levels of cAMP in neurons. Other representative agents include growth factors such as nerve growth factor, heregulin, laminin, neurotrophin, brain-derived neurotrophic factor; glia-derived neurotrophic factor, ciliary neurotrophic factor, neuregulin, tenascin, fibronectin, netrin-1, neurturin, and neural cell adhesion molecule. Still additional agents include neurotransmitters and neuropeptides, including modulators of the cholinergic, noradrenergic, serotonergic, GABAergic, glutaminergic, or peptidergic systems. TNFRSF19 modulators may also be administered in combination with cellular therapies. See e.g., U.S. Pat. No. 6,787,356.

For combination therapies, a TNFRSF19 modulator and an additional therapeutic are administered within any time frame suitable for performance of the intended therapy. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s). The administration of the TNFRSF19 modulator and a second therapeutic agent preferably elicits greater neurite outgrowth than administration of either alone.

For human subjects, the therapeutically effective dose of TNFRSF19 modulator may be estimated initially either in cell culture assays or in animal models, usually in rodents, rabbits, dogs, pigs, and/or or primates. The animal model may also be used to determine the appropriate concentration range and route of administration. Such information may then be used to determine useful doses and routes for administration in humans. Typically a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine.

For additional guidance regarding formulation, dose, administration regimen, and measurable therapeutic outcomes, see Berkow et al. (2000) The Merck Manual of Medical Information, Merck & Co., Inc., Whitehouse Station, N.J.; Ebadi (1998) CRC Desk Reference of Clinical Pharmacologv, CRC Press, Boca Raton, Fla; Gennaro (2000) Remington: The Science and Practice of Pharmacy, Lippincott, Williams & Wilkins, Philadelphia, Pa.; Katzung (2001) Basic & Clinical Pharmacology, Lange Medical Books/McGraw-Hill Medical Pub. Div., New York; Hardman et al. (2001) Goodman & Gilman's the Pharmacological Basis of Therapeutics, The McGraw-Hill Companies, Columbus, Ohio; Speight & Holford (1997) Avery's Drug Treatment: A Guide to the Properties. Choices, Therapeutic Use and Economic Value of Drugs in Disease Management, Lippincott, Williams, & Wilkins, Philadelphia, Pa.

### EXAMPLES

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention.

### Example 1

Cloning and Characterization of Rat TNFRSF19

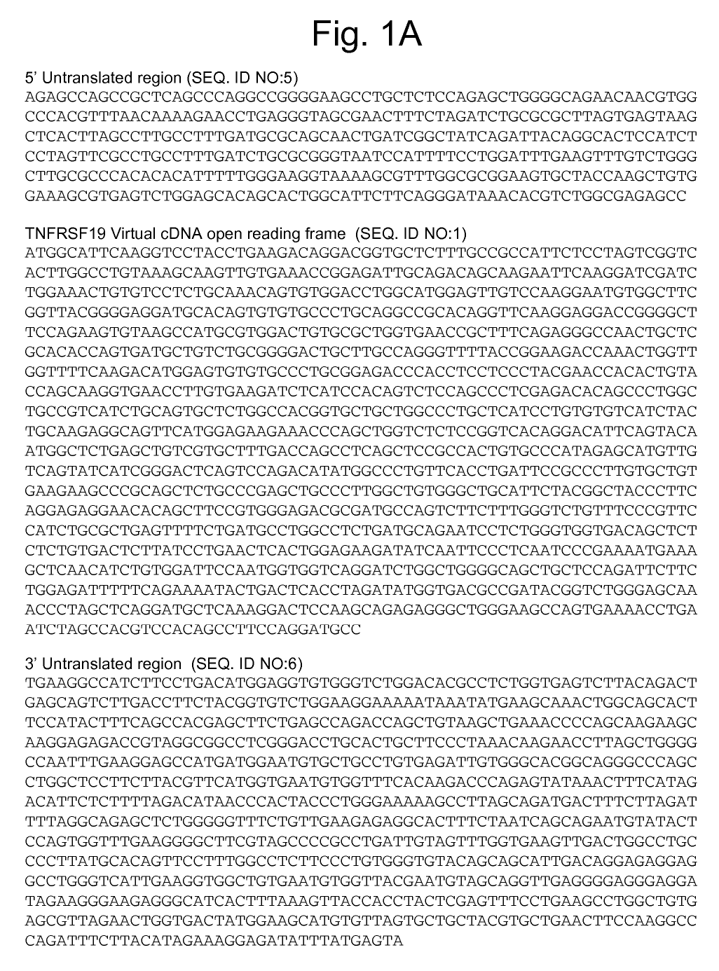

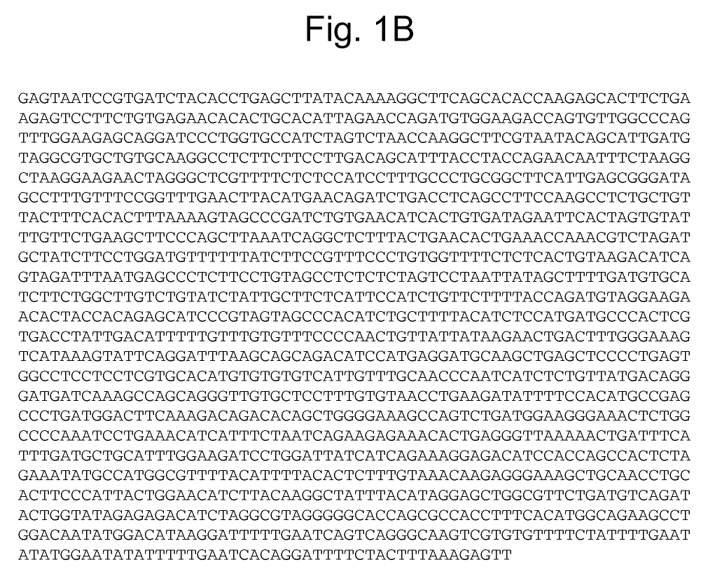

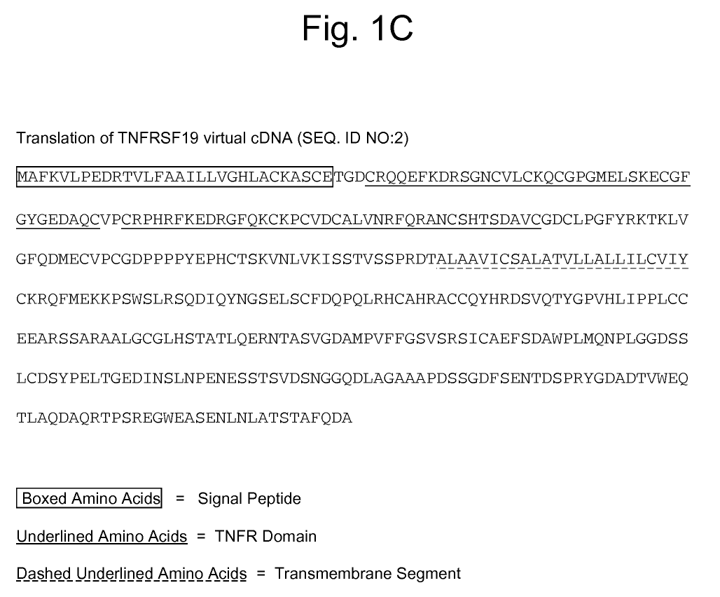

Rat TNFRSF19 virtual cDNA was assembled based on mouse TROY (GenBank Accession No. NM_{—}013869.3) and rat genomic sequence obtained from Celera Genomics (Rockville, Md.). The virtual rat TNFRSF19 cDNA (SEQ ID NO:1) and 5′ and 3′ untranslated regions (SEQ ID NOs: 5 and 6, respectively) are set forth in FIG. 1. The following oligonucleotide primers were prepared based upon the virtual cDNA sequence:

| | |
|---|---|
| sense: | |
| 5′-CGCGGATCCATGGCATTCAAGGTCCTACC-3′ | (SEQ ID NO: 7) |
| antisense: | |
| 5′-CCCAAGCTTTCCGGCATCCTGGAAGGCTG-3′. | (SEQ ID NO: 8) |

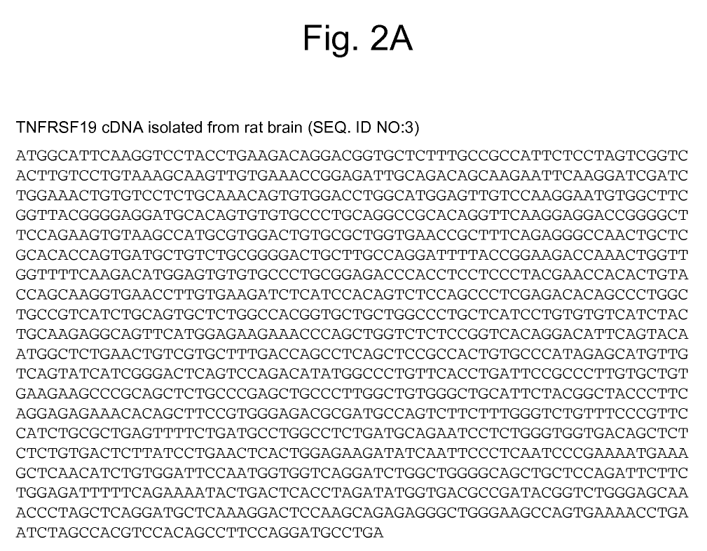

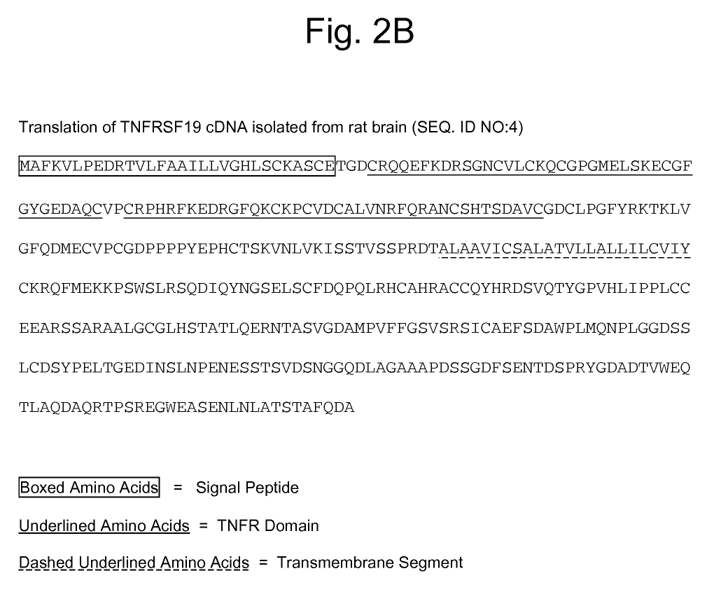

A cDNA clone was isolated following PCR amplification using the above-noted primers, rat brain QUICK-CLONE™ cDNA (Clontech of Mountainview, California) derived from Sprague-Dawley rats as template, and the ADVANTAGE®-GC 2 PCR kit (Clontech). The 1247 base-pair amplified product was sub-cloned into pCR2.1 vector of the pCR-Blunt 11 TOPO® cloning kit (Invitrogen of Carlsbad, Calif.). The nucleotide sequence of the cDNA was determined by automated cycle sequencing (Applied Biosystems) and is set for in FIG. 2 (SEQ ID NO:3).

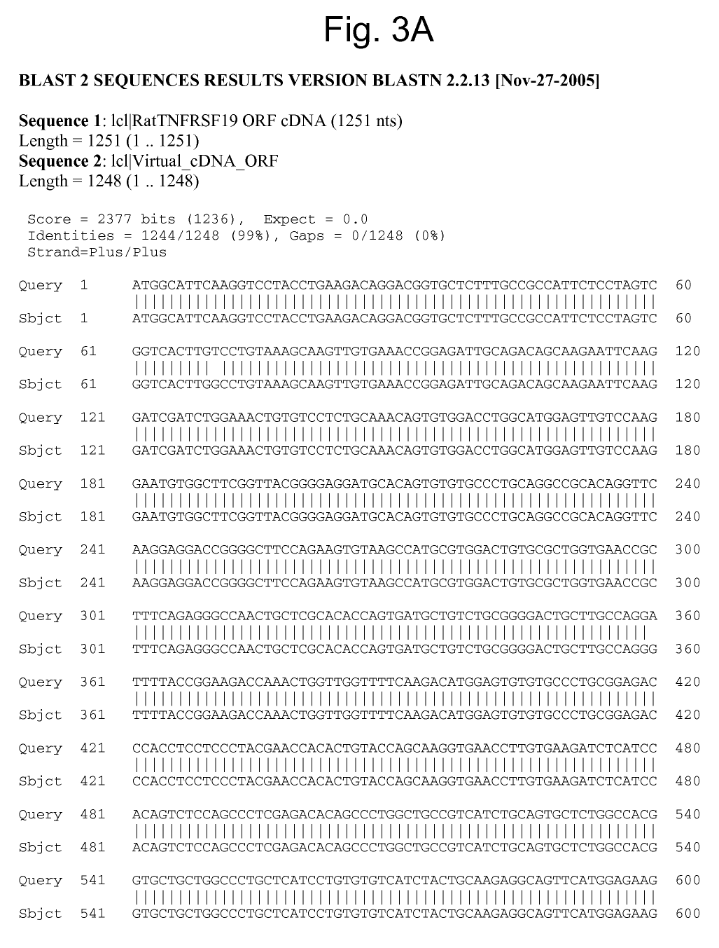

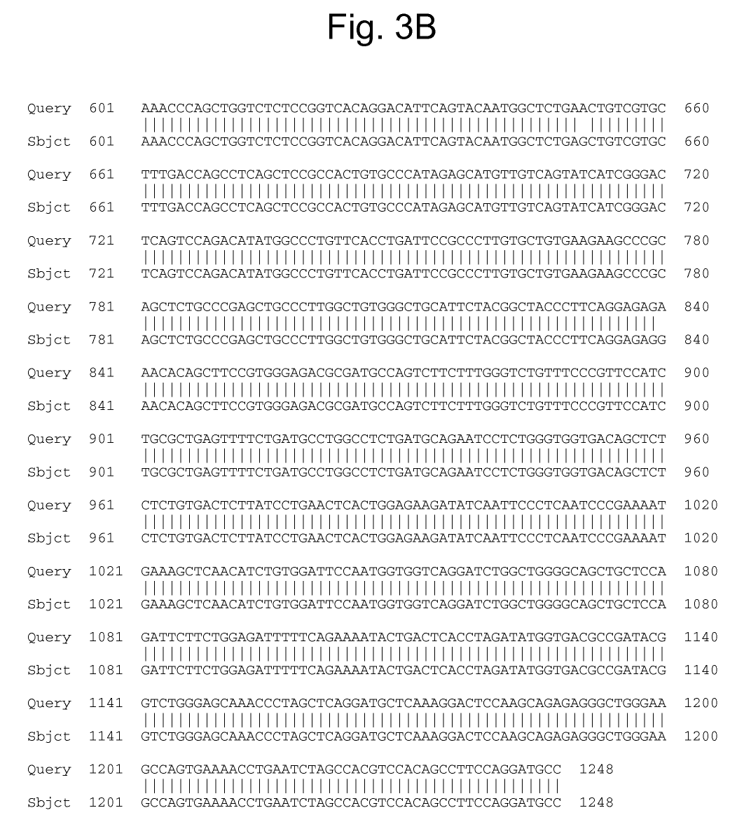

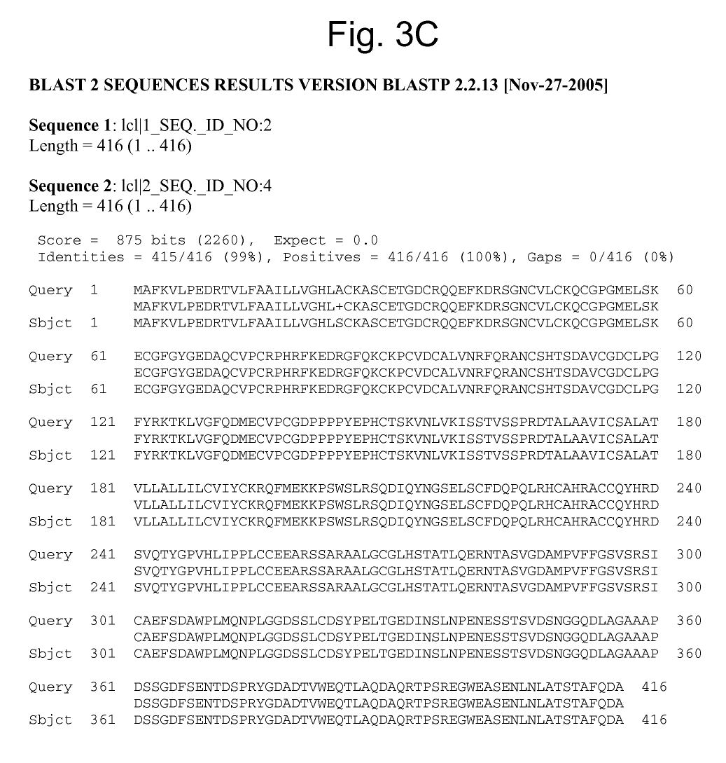

The cloned rat TNFRSF19 (SEQ ID NO:3) includes four nucleotide differences when compared to the reference rat TNFRSF19 virtual sequence (SEQ ID NO:1). See alignment in FIGS. 3A-3B. There is only one amino acid difference, i.e., an alanine is present in the virtual sequence at position 24, whereas a serine is present in the cloned TNFRSF19 at this same position. This amino acid difference is most likely a polymorphism since A24 is presented in murine sequence while S24 is in human sequence. See alignment in FIGS. 3C-3D.

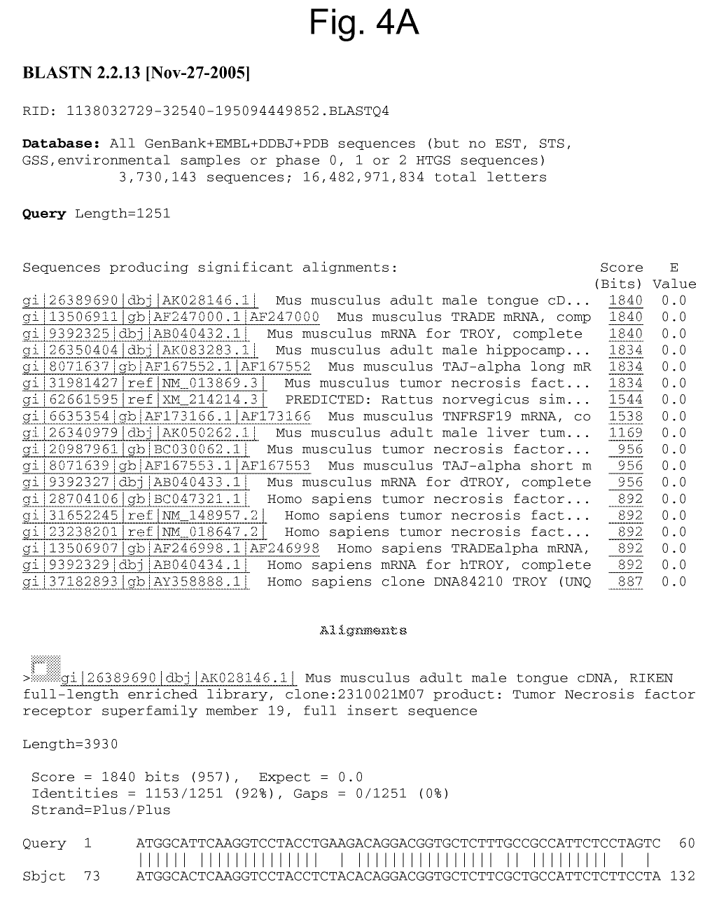

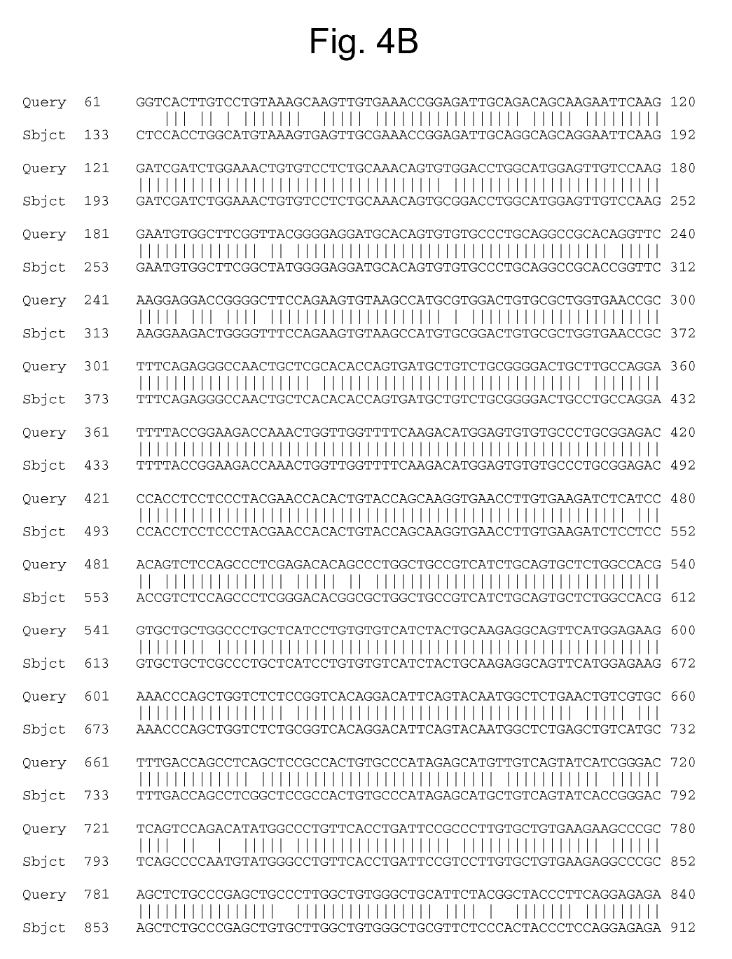

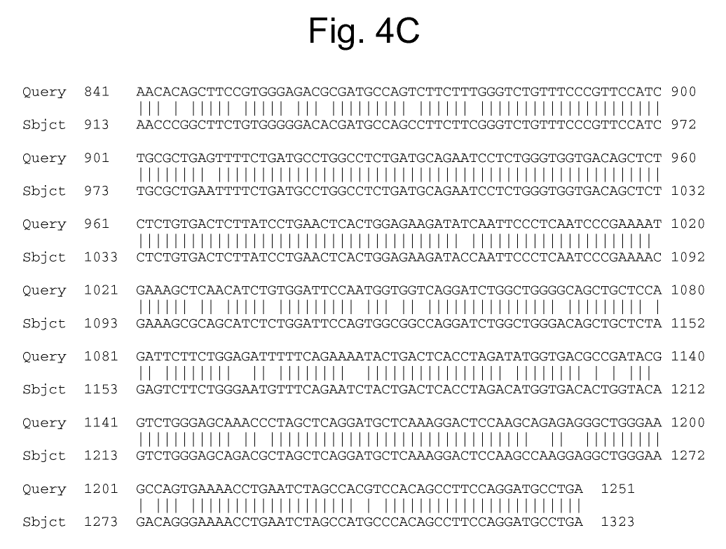

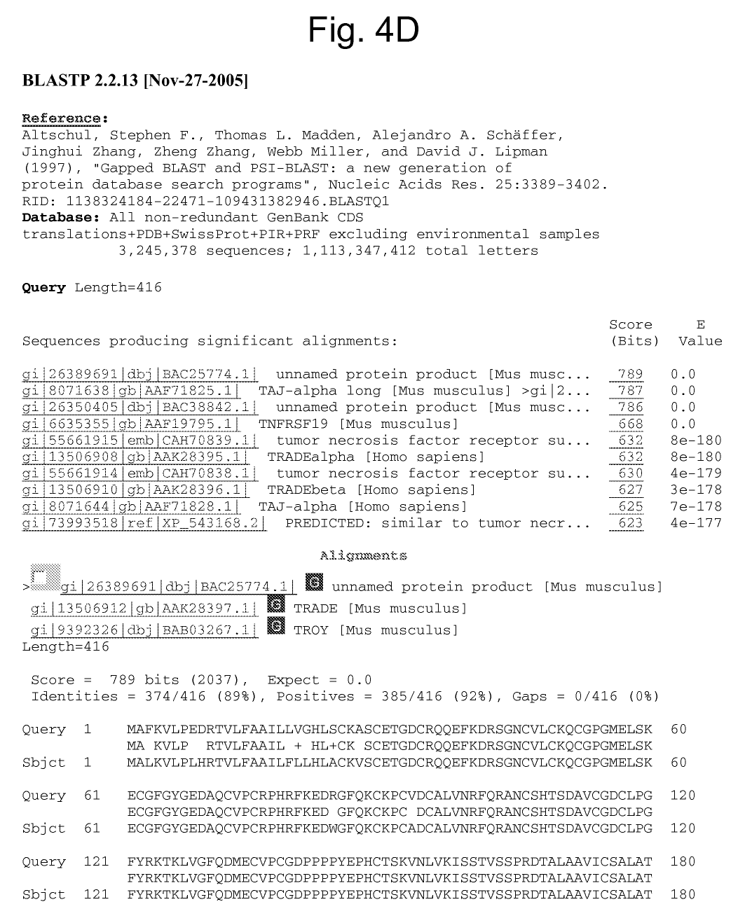

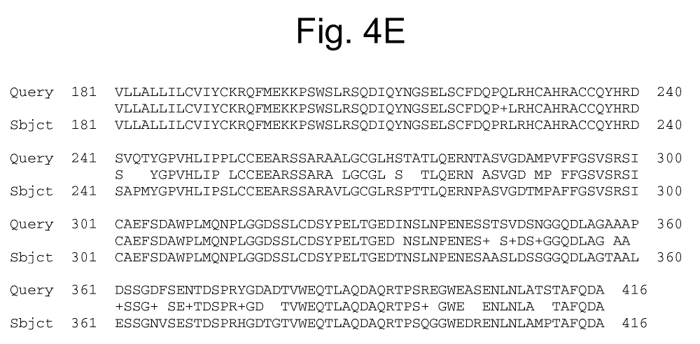

To assess the novelty of the rat TNFRSF19 cDNAs, BLASTp searches (for protein query sequences) were conducted using default parameters of Expect=10, Word Size=3, a low complexity filter, and the BLOSUM62 matrix, permitting gap costs of existence=11, and extension=1. BLASTn searches (for nucleotide query sequences) were conducted using default parameters of Expect=10, Word Size=11, and a low complexity filter. BLAST search results are reported as a list of sequences related to the query sequence, ranked in order of E value, which is an indicator of the statistical significance of matches identified in the database. Sequences most closely related to the rat TNFRSF19 query sequences are identified in Table 1. The BLAST results and an alignment of each query sequence with the most closely related subject sequence are shown in FIGS. 4A-4E.

TABLE 1
BLAST Analysis

| | Identity (%) of | | |
|---|---|---|---|
| | Closest Subject | | |
| Query Sequence | Sequence | Description of Closest Subject Sequence | FIG. |
| rat TNFRSF19 | 92% | gi\|26389690\|dbj\|AK028146.1\|: Mus | 4A-4C |
| cDNA | | musculus adult male tongue cDNA, | |
| | | RIKEN full-length enriched library, | |
| | | clone: 2310021M07 product: Tumor | |
| | | Necrosis factor receptor superfamily | |
| | | member 19 | |
| | | gi\|13506911\|gb\|AF247000.1\|AF247000: | |
| | | Mus musculus TRADE mRNA | |
| | | gi\|9392325\|dbj\|AB040432.1\|: Mus | |
| | | musculus mRNA for TROY | |
| rat TNFRSF19 | 90% | gi\|26389691\|dbj\|BAC25774.1; unnamed | 4D-4E |
| protein | | protein product [Mus musculus] | |
| | | gi\|8071638\|gb\|AAF1825.1; TAJ-alpha | |
| | | long [Mus musculus] | |
| | | gi\|26350405\|dbj\|BAC38842.1; unnamed | |
| | | protein product [Mus musculus] | |
| | | gi\|6635355\|gb\|AAF19795.1; TNFRSF19 | |
| | | [Mus musculus] | |

Two predicted rat TNFRSF19 mRNAs were also identified in the BLAST searches (GenBank Accession Nos. XM_{—}214214 and XM_{—}341893). Both sequences were predicted by automated computational analysis of a genomic sequence. Pairwise alignments of the rat TNFRSF19 query sequence and GenBank Accession No. XM_{—}214214 or XM_{—}341983 showed substantial dissimilarity in overall structure and identity limited to only a portion of the sequence.

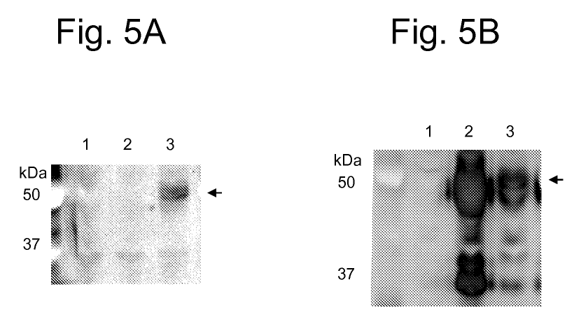

To confirm that rat TNFRSR19 could be expressed, the rat TNFRSF19 cDNA was subcloned into a mammalian expression vector pcDNA3.1/Myc-His(−)A (Invitrogen). The stop codon was changed to a glycine for the purpose of expressing rat TNFRSF19 as a tagged protein. This cDNA was transfected into CHO-K1 cells using LIPOFECTAMINE™ 2000 (Invitrogen) over a period of 24 hours. Cells lysates were prepared and used for Western blot analysis with anti-mouse TROY (Santa Cruz Biotechnology of Santa-Cruz, Calif.) and anti-Myc antibodies (Invitrogen). Rat TNFRSF19 having a molecular weight of about 50 kDa was detected. See FIGS. 5A-5B.

### Example 2

Interaction of Rat TNFRSF19 with NGR and Lingo-1

To assess the interaction of rat TNFRSF19 with NgR and LINGO-1, CHO-K1 cells are transfected with vectors expressing full-length rat TNFRSF19 (Myc-tagged), human or rat NgR (including any one of NgR1, NgR2, or NgR3), and human or rat LINGO-1. After sufficient time for formation of a complex, cells are harvested and lysed in 1 ml lysis buffer (50 mM HEPES [pH 7.5], 150 mM NaCl, 1.5 mM MgCl₂, 1 mM EGTA, 1% TRITON-X® 100 and 10% glycerol) for 30 minutes at 4° C. The lysates are centrifuged at 14,000×g for 15 minutes. The supernatants are recovered and incubated with Protein A/G-Sepharose beads (Santa Cruz Biotechnology, Inc.) at 4° C.for 1 hour. They are then incubated at 4° C. for an additional 1 hour with anti-NgR antibody, anti-mouse TNFRSF19 antibody, or anti-Myc antibody plus Protein A/G-Sepharose beads. The beads are washed three times with lysis buffer and boiled in Laemmli sample buffer. Samples are resolved using 4%-20% SDS-PAGE and analyzed by Western blotting with anti-NgR antibody, anti-mouse TNFRSF19 antibody, or anti-Myc antibody. Formation of a NgR complex and presence of rat TNRSF19 in the complex is observed upon detection of NgR in samples precipitated with anti-mouse TNFRSF19 or with anti-Myc antibodies, or upon detection of TNFRSF19 in samples precipitated with anti-NgR antibody. TNFRSF19 residues required for binding to NgR and/or LINGO-1 are identified by performing similar experiments using truncated TNFRSF19 proteins tagged with Myc or TNFRSF19 proteins having mutated residues.

### Example 3

Assay of RHO Activation

To assess if the interactions between rat TNFRSF19, NgR (including any one of NgR1, NgR2, or NgR3), and LINGO-1 are indicative of a functional MAIF receptor complex, CHO-K1 cells are transfected with vectors expressing full-length rat TNFRSF19 (Myc-tagged), human or rat NgR, and human or rat LINGO-1. After sufficient time for formation of a complex, the transfected cells are treated with 10 μg/ml of each of AP-OMgp, GST-Nogo-66 or other appropriate ligand, MAG-Fc, or control protein. Cells are then lysed in 50 mM Tris-HCl (pH 7.5), 1% TRITON-X® 100, 0.5% sodium deoxycholate, 0.1% SDS, 500 mM NaCl, 10 mM MgCl₂, plus protease inhibitors. RhoA bound to GTP and total RhoA proteins are then detected by Western blotting using anti-RhoA mAb (Santa Cruz Biotechnology, Inc.) essentially as described by Mi et al. (2004) Nat. Neurosci. 7:221-228. Activation of RhoA Is observed as increased levels of RhoA bound to GTP.

### Example 4

TNFRSF19 Knockout Rats

To assess a biological role of rat TNFRSF19, rats are prepared so as to disrupt the TNFRSF19 gene. Representative methods for producing transgenic rats can be found, for example, in Haag et al. (2003) Cancer Res. 63(18):5808-5812 and U.S. Pat. No. 5,489,742, which are incorporated by reference herein in their entirety. For preparation of appropriate constructs for preparing knock-out or knock-in rats, a genomic map of the rat TNFRSF19 loci is depicted in FIG. 6.

To analyze the effects of TNFRSR19 deletion on the response of neurons to MAIFs, neurons from TNFRSR19 knockout and wild type rats are plated onto myelin inhibitor-coated slides, and neurite outgrowth is measured 1 day later. Primary cerebellar granule (CG) neuron cultures and dorsal root ganglion (DRG) neuron cultures are prepared using 4-well culture slides (Labtek of Campbell, Calif.) coated with 0.1 mg/ml poly-D-lysine (Sigma-Aldrich of St. Louis, Mo.) before spotting with 1 μg/3 μl AP-Nogo-66, or 1 μg/3 μl AP-OMgp, or 1 μg/3 μl AP-TNFRSF or control-Fc (human IgG1). The slides are then dried at room temperature for 2 hours prior to plating of cells. CG neurons (approximately 1.5×10⁵ cells per well) or DRG neurons (approximately 5000 cells per well) from P7 rats are dissociated, seeded onto the slides, and incubated at 37° C. in 5% CO₂ for 16 hours. The slides are fixed in 4% paraformaldehyde/20% sucrose and stained with anti-βIII-tubulin (clone TUJI) (Covance Inc. of Princeton, N.J.). Average neurite length is quantified by measuring lengths of individual neurites from multiple representative microscope fields (e.g., 300 to 500 cells per sample) and dividing by the total number of cells counted. Alternatively, cell lysates are used to prepare Western blots, and βIII-tubulin levels are quantified by densitometry.

### Example 5

Assay of Neurite Outgrowth Following Disruption of TNFSRF19 Using Single-stranded Interfering RNAs

Single-stranded interfering RNAs (siRNA) directed against TNFSRF19 are designed using criteria set out by Elbashir et al. (2001) Nature 411(6836):494-498. Selected sequences are subjected to a BLAST search to ensure no significant homology with other genes. The sense siRNA template includes an AA dimer at the 5′ end followed by the 19 nucleotide complementary to the target sequence. The 3′ end of both the sense and antisense templates include an eight nucleotide sequence corresponding to the complementary sequence of the T7 promoter primer required for efficient transcription of the siRNA. Deprotected and desalted oligonucleotide templates and control scrambled sequences are chemically synthesized.

CG and DRG neuron cultures are prepared as in Example 4. For transfection of siRNAs into cultured neurons, a NUCLEOFECTOR® kit is used according to the vendor's instructions (amaxa Inc. of Gaithersburg, Md). Neurite outgrowth is measured as described in Example 4.

To assess neuronal viability following siRNA-mediated TNFRSF19 knockdown, the number of βIII-tubulin-positive neurons are counted. The mean number of viable neurons in siRNA-treated versus control cultures is determined.

### Example 6

Neurite Outgrowth in Response to a TNFRSF19 Inhibitor

To assess the function of TNFRSF19 inhibitors identified as described herein, activation of RhoA and neurite outgrowth are tested following exposure of cultured neurons to a TNFRSF19 inhibitor. RhoA activation is measured as described in Example 3 following addition of a TNFRSF19 inhibitor to the cells transfected with TNFRSF19, NgR (including any one of NgR1, NgR2, or NgR3), and LINGO-1 or to CG or DRG neurons. Neurite outgrowth is measured as described in Example 4 following addition of a TNFRSF19 inhibitor to the culture medium.

### Example 7

Corticospinal Tract Regeneration Assay

To further test the effect of TNFRSF19 inhibitors on neurite outgrowth in vivo, nerve regeneration is assayed following administration of a TNFRSF19 inhibitor to an animal having a spinal cord injury. See e.g., Guest et al. (1997) J. Neurosci. Res. 50(5):888-905. A spinal cord injury is created by transecting the midthoracic spinal cord of an adult nude rat. Human Schwann cell grafts are placed to span the transection and thereby facilitate regeneration. A TNFRSF19 inhibitor is incorporated into a fibrin glue and placed in the same region.

Approximately thirty-five days after grafting, response to the TNFRSF19 inhibitor is evaluated qualitatively by looking for regenerated axon fibers in or beyond grafts. New axon fibers are visualized by anterograde tracing from the motor cortex using tracer molecules (e.g., dextran amine tracers such as Fluororuby and biotinylated dextran amine). The response is quantified by constructing camera lucida composites to determine the sprouting index, the position of the maximum termination density rostral to a host/graft interface (defined by staining with anti-glial acidic fibrillary protein (GFAP)), and the longitudinal spread of bulbous end terminals. The latter two measures provide information about axonal die-back. In control animals receiving a human Schwann cell graft only (e.g., without a TNFRSF19 inhibitor), axons do not enter the Schwann cell graft and undergo axonal die-back. The presence of axon outgrowths in the graft indicates disinhibition of neurite outgrowth by a TNFRSF19 inhibitor.

### Example 8

Peripheral Nerve Regeneration Assay

To assess the effect of TNFRSF19 inhibitors on neurite outgrowth in vivo, nerve regeneration is assayed following administration of a TNFRSF19 inhibitor to an animal having a peripheral nerve injury. The sciatic nerves of rats are transected at mid-thigh and guide tubes containing test agents with and without guiding filaments sutured over distances of approximately 2 mm to the end of the nerves. In each experiment, the other end of the guide tube is left open. This model simulates a severe nerve injury in which no contact with the distal end of the nerve is present.

TNFRSF19 inhibitors are incorporated in implantable devices (e.g., as described in U.S. Pat. No. 5,656,605) and tested for promotion nerve regeneration. Surgical-grade silicon rubber tubes (I.D. 1.5 mm) are prepared with or without guiding filaments (four 10-0 monofilament nylon) and loaded with TNFRSF19 inhibitors and binding agents suitable for forming a matrix (e.g., BIOMATRIX™ available from Biomedical Technologies, Inc. of Stoughton, Mass.).

After about four weeks, the distance of regeneration of axons within the guide tube is tested in surviving animals using a functional pinch test. In this test, the guide tube is pinched with fine forceps to mechanically stimulate sensory axons. Testing is initiated at the distal end of the guide tube and advanced proximally until muscular contractions are noted in the lightly anesthetized animal. Nerve regeneration is measured as the distance from the point of nerve transection point at which stimulation elicits a sensory response.

For histological analysis, the guide tube containing the regenerated nerve is preserved with a fixative. Cross sections are prepared at a point approximately 7 mm from the transection site. The diameter of the regenerated nerve and the number of myelinated axons observable at this point are used as additional indicators of the degree of nerve regeneration.

1. An isolated TNFRSF19 nucleic acid comprising:
(a) an open reading frame encoding a TNFRSF19 protein comprising an amino acid sequence at least 91% identical to SEQ ID NO:2 or 4;
(b) an open reading frame encoding a TNFRSF19 protein comprising an amino acid sequence of SEQ ID NO:2 or 4;
(c) a nucleotide sequence at least 93% identical to SEQ ID NO: 1 or 3;
(d) a nucleotide sequence of SEQ ID NO:1 or 3;
(e) a nucleic acid that specifically hybridizes to the complement of SEQ ID NO:1 or 3 under stringent hybridization conditions; or
(f) a nucleotide sequence that is the complement of any one of (a)-(e).

2. The isolated nucleic acid of claim 1, which encodes a TNFRSF19 protein that forms a receptor complex comprising NgR.

3. The isolated nucleic of claim 1, which encodes a TNFRSF19 protein that forms a receptor complex that blocks neurite outgrowth.

4. The isolated nucleic of claim 1, which encodes a TNFRSF19 protein that disinhibits neurite outgrowth.

5. A vector comprising the nucleic acid of claim 1.

6. A host cell which expresses the vector of claim 5.

7. An isolated TNFRSF19 protein comprising:
(a) an amino acid sequence at least 91% identical to SEQ ID NO:2 or 4; or
(b) an amino acid sequence of SEQ ID NO:2 or 4.

8. The isolated TNFRSF19 protein of claim 7, which forms a receptor complex comprising NgR.

9. The isolated TNFRSF19 protein of claim 7, which forms a receptor complex that blocks neurite outgrowth.

10. The isolated TNFRSF19 protein of claim 7, which disinhibits neurite outgrowth.

11. An antibody or antibody fragment which specifically binds to the isolated TNFRSF19 protein of claim 7, and which disinhibits neurite outgrowth.

12. The antibody of claim 11, which is a chimeric, humanized, or human antibody.

13. A genetically modified rat comprising a mutation in a nucleotide sequence encoding a TNFSRF19 protein of claim 7, wherein the mutation alters (i) expression of a TNFRSF19 protein, or (ii) function of a TNFRSF19 protein in forming a receptor complex comprising TNFRSF19 and NgR.

14. A method for identifying a binding partner of a TNFRSF19 protein, the method comprising the steps of:
(a) providing a TNFRSF19 protein comprising (i) an amino acid sequence at least 91% identical to SEQ ID NO:2 or 4; or (ii) an amino acid sequence of SEQ ID NO:2 or 4;
(b) providing a one or more test agents to the TNFRSF19 protein under conditions sufficient for binding;
(c) assaying binding of a test agent to the isolated TNFRSF19 protein; and
(d) selecting a test agent that demonstrates specific binding to the TNFRSF19 protein.

15. The method of claim 14, wherein the one or more test agents is a peptide, a protein, an oligomer, a nucleic acid, a small molecule, or an antibody.

16. A method for promoting neurite outgrowth in vitro or in vivo comprising contacting neurons with a TNFRSF19 binding partner identified according to the method of claim 14.

17. A method for identifying agents for disinhibition of neurite outgrowth comprising the steps of:
(a) providing a TNFRSF19 protein under conditions for formation of a receptor complex comprising NgR, wherein the TNFRSF19 protein comprises (i) an amino acid sequence at least 91% identical to SEQ ID NO:2 or 4; or (ii) an amino acid sequence of SEQ ID NO:2 or 4;
(b) providing one or more test agents or a control agent to the TNFRSF19 protein and NgR of (a);
(c) allowing sufficient time for formation of a receptor complex;
(d) assaying (i) formation of a receptor complex, (ii) capacity of the receptor complex of (c) to activate RhoA; or (iii) capacity of the receptor complex of (c) to inhibit neurite outgrowth; and
(e) selecting a test agent that shows (i) altered formation of a receptor complex in the presence of the agent as compared to a control agent, (ii) altered capacity of the receptor complex of (c) to activate RhoA when in the presence of the test agent as compared to a control agent; or (iii) altered capacity of the receptor complex of (c) to inhibit neurite outgrowth when in the presence of the test agent as compared to a control agent.

18. The method of claim 17, wherein the one or more test agents is a peptide, a protein, an oligomer, a nucleic acid, a small molecule, or an antibody.

19. A method for promoting neurite outgrowth in vitro or in vivo comprising contacting neurons with a TNFRSF19 binding partner identified according to the method of claim 14.

20. A method for promoting neurite outgrowth in vitro or in vivo comprising contacting neurons with a TNFRSF19 modulator identified according to the method of claim 17.


SEQUENCE LISTING (6 sequences)

SEQ ID NO: 1 - DNA, 1248 bp, Sprague-Dawley rat
atggcattcaaggtcctacctgaagacaggacggtgctctttgccgccattctcctagtcggtcacttggcctgtaaagcaagttgtgaaaccggagattgcagacagcaagaattcaaggatcgatctggaaactgtgtcctctgcaaacagtgtggacctggcatggagttgtccaaggaatgtggcttcggttacggggaggatgcacagtgtgtgccctgcaggccgcacaggttcaaggaggaccggggcttccagaagtgtaagccatgcgtggactgtgcgctggtgaaccgctttcagagggccaactgctcgcacaccagtgatgctgtctgcggggactgcttgccagggttttaccggaagaccaaactggttggttttcaagacatggagtgtgtgccctgcggagacccacctcctccctacgaaccacactgtaccagcaaggtgaaccttgtgaagatctcatccacagtctccagccctcgagacacagccctggctgccgtcatctgcagtgctctggccacggtgctgctggccctgctcatcctgtgtgtcatctactgcaagaggcagttcatggagaagaaacccagctggtctctccggtcacaggacattcagtacaatggctctgagctgtcgtgctttgaccagcctcagctccgccactgtgcccatagagcatgttgtcagtatcatcgggactcagtccagacatatggccctgttcacctgattccgcccttgtgctgtgaagaagcccgcagctctgcccgagctgcccttggctgtgggctgcattctacggctacccttcaggagaggaacacagcttccgtgggagacgcgatgccagtcttctttgggtctgtttcccgttccatctgcgctgagttttctgatgcctggcctctgatgcagaatcctctgggtggtgacagctctctctgtgactcttatcctgaactcactggagaagatatcaattccctcaatcccgaaaatgaaagctcaacatctgtggattccaatggtggtcaggatctggctggggcagctgctccagattcttctggagatttttcagaaaatactgactcacctagatatggtgacgccgatacggtctgggagcaaaccctagctcaggatgctcaaaggactccaagcagagagggctgggaagccagtgaaaacctgaatctagccacgtccacagccttccaggatgcc

SEQ ID NO: 2 - protein, 416 aa, Sprague-Dawley rat
  SIGNAL_PEPTIDE (1)..(30)
  TNFR_Domain (34)..(72)
  TNFR_Domain (75)..(114)
  Transmembrane_Domain (169)..(192)
MAFKVLPEDRTVLFAAILLVGHLACKASCETGDCRQQEFKDRSGNCVLCKQCGPGMELSKECGFGYGEDAQCVPCRPHRFKEDRGFQKCKPCVDCALVNRFQRANCSHTSDAVCGDCLPGFYRKTKLVGFQDMECVPCGDPPPPYEPHCTSKVNLVKISSTVSSPRDTALAAVICSALATVLLALLILCVIYCKRQFMEKKPSWSLRSQDIQYNGSELSCFDQPQLRHCAHRACCQYHRDSVQTYGPVHLIPPLCCEEARSSARAALGCGLHSTATLQERNTASVGDAMPVFFGSVSRSICAEFSDAWPLMQNPLGGDSSLCDSYPELTGEDINSLNPENESSTSVDSNGGQDLAGAAAPDSSGDFSENTDSPRYGDADTVWEQTLAQDAQRTPSREGWEASENLNLATSTAFQDA

SEQ ID NO: 3 - DNA, 1251 bp, Sprague-Dawley rat
atggcattcaaggtcctacctgaagacaggacggtgctctttgccgccattctcctagtcggtcacttgtcctgtaaagcaagttgtgaaaccggagattgcagacagcaagaattcaaggatcgatctggaaactgtgtcctctgcaaacagtgtggacctggcatggagttgtccaaggaatgtggcttcggttacggggaggatgcacagtgtgtgccctgcaggccgcacaggttcaaggaggaccggggcttccagaagtgtaagccatgcgtggactgtgcgctggtgaaccgctttcagagggccaactgctcgcacaccagtgatgctgtctgcggggactgcttgccaggattttaccggaagaccaaactggttggttttcaagacatggagtgtgtgccctgcggagacccacctcctccctacgaaccacactgtaccagcaaggtgaaccttgtgaagatctcatccacagtctccagccctcgagacacagccctggctgccgtcatctgcagtgctctggccacggtgctgctggccctgctcatcctgtgtgtcatctactgcaagaggcagttcatggagaagaaacccagctggtctctccggtcacaggacattcagtacaatggctctgaactgtcgtgctttgaccagcctcagctccgccactgtgcccatagagcatgttgtcagtatcatcgggactcagtccagacatatggccctgttcacctgattccgcccttgtgctgtgaagaagcccgcagctctgcccgagctgcccttggctgtgggctgcattctacggctacccttcaggagagaaacacagcttccgtgggagacgcgatgccagtcttctttgggtctgtttcccgttccatctgcgctgagttttctgatgcctggcctctgatgcagaatcctctgggtggtgacagctctctctgtgactcttatcctgaactcactggagaagatatcaattccctcaatcccgaaaatgaaagctcaacatctgtggattccaatggtggtcaggatctggctggggcagctgctccagattcttctggagatttttcagaaaatactgactcacctagatatggtgacgccgatacggtctgggagcaaaccctagctcaggatgctcaaaggactccaagcagagagggctgggaagccagtgaaaacctgaatctagccacgtccacagccttccaggatgcctga

SEQ ID NO: 4 - protein, 416 aa, Sprague-Dawley rat
  Signal_Peptide (1)..(30)
  TNFR_Domain (34)..(72)
  TNFR_Domain (75)..(114)
  Transmembrane_Domain (169)..(192)
MAFKVLPEDRTVLFAAILLVGHLSCKASCETGDCRQQEFKDRSGNCVLCKQCGPGMELSKECGFGYGEDAQCVPCRPHRFKEDRGFQKCKPCVDCALVNRFQRANCSHTSDAVCGDCLPGFYRKTKLVGFQDMECVPCGDPPPPYEPHCTSKVNLVKISSTVSSPRDTALAAVICSALATVLLALLILCVIYCKRQFMEKKPSWSLRSQDIQYNGSELSCFDQPQLRHCAHRACCQYHRDSVQTYGPVHLIPPLCCEEARSSARAALGCGLHSTATLQERNTASVGDAMPVFFGSVSRSICAEFSDAWPLMQNPLGGDSSLCDSYPELTGEDINSLNPENESSTSVDSNGGQDLAGAAAPDSSGDFSENTDSPRYGDADTVWEQTLAQDAQRTPSREGWEASENLNLATSTAFQDA

SEQ ID NO: 5 - DNA, 383 bp, Sprague-Dawley rat
agagccagccgctcagcccaggccggggaagcctgctctccagagctggggcagaacaacgtggcccacgtttaacaaaagaacctgagggtagcgaactttctagatctgcgcgcttagtgagtaagctcacttagccttgcctttgatgcgcagcaactgatcggctatcagattacaggcactccatctcctagttcgcctgcctttgatctgcgcgggtaatccattttcctggatttgaagtttgtctgggcttgcgcccacacacatttttgggaaggtaaaagcgtttggcgcggaagtgctaccaagctgtggaaagcgtgagtctggagcacagcactggcattcttcagggataaacacgtctggcgagagcc

SEQ ID NO: 6 - DNA, 2387 bp, Sprague-Dawley rat
tgaaggccatcttcctgacatggaggtgtgggtctggacacgcctctggtgagtcttacagactgagcagtcttgaccttctacggtgtctggaaggaaaaataaatatgaagcaaactggcagcacttccatactttcagccacgagcttctgagccagaccagctgtaagctgaaaccccagcaagaagcaaggagagaccgtaggcggcctcgggacctgcactgcttccctaaacaagaaccttagctggggccaatttgaaggagccatgatggaatgtgctgcctgtgagattgtgggcacggcagggcccagcctggctccttcttacgttcatggtgaatgtggtttcacaagacccagagtataaactttcatagacattctcttttagacataacccactaccctgggaaaaagccttagcagatgactttcttagattttaggcagagctctgggggtttctgttgaagagaggcactttctaatcagcagaatgtatactccagtggtttgaaggggcttcgtagccccgcctgattgtagtttggtgaagttgactggcctgccccttatgcacagttcctttggcctcttccctgtgggtgtacagcagcattgacaggagaggaggcctgggtcattgaaggtggctgtgaatgtggttacgaatgtagcaggttgaggggagggaggatagaagggaagagggcatcactttaaagttaccacctactcgagtttcctgaagcctggctgtgagcgttagaactggtgactatggaagcatgtgttagtgctgctacgtgctgaacttccaaggcccagatttcttacatagaaaggagatatttatgagtagagtaatccgtgatctacacctgagcttatacaaaaggcttcagcacaccaagagcacttctgaagagtccttctgtgagaacacactgcacattagaaccagatgtggaagaccagtgttggcccagtttggaagagcaggatccctggtgccatctagtctaaccaaggcttcgtaatacagcattgatgtaggcgtgctgtgcaaggcctcttcttccttgacagcatttacctaccagaacaatttctaaggctaaggaagaactagggctcgttttctctccatcctttgccctgcggcttcattgagcgggatagcctttgtttccggtttgaacttacatgaacagatctgacctcagccttccaagcctctgctgttactttcacactttaaaagtagcccgatctgtgaacatcactgtgatagaattcactagtgtatttgttctgaagcttcccagcttaaatcaggctctttactgaacactgaaaccaaacgtctagatgctatcttcctggatgttttttatcttccgtttccctgtggttttctctcactgtaagacatcagtagatttaatgagccctcttcctgtagcctctctctagtcctaattatagcttttgatgtgcatcttctggcttgtctgtatctattgcttctcattccatctgttcttttaccagatgtaggaagaacactaccacagagcatcccgtagtagcccacatctgcttttacatctccatgatgcccactcgtgacctattgacatttttgtttgtgtttccccaactgttattataagaactgactttgggaaagtcataaagtattcaggatttaagcagcagacatccatgaggatgcaagctgagctcccctgagtggcctcctcctcgtgcacatgtgtgtgtcattgtttgcaacccaatcatctctgttatgacagggatgatcaaagccagcagggttgtgctcctttgtgtaacctgaagatattttccacatgccgagccctgatggacttcaaagacagacacagctggggaaagccagtctgatggaagggaaactctggccccaaatcctgaaacatcatttctaatcagaagagaaacactgagggttaaaaactgatttcatttgatgctgcatttggaagatcctggattatcatcagaaaggagacatccaccagccactctagaaatatgccatggcgttttacattttacactctttgtaaacaagagggaaagctgcaacctgcacttcccattactggaacatcttacaaggctatttacataggagctggcgttctgatgtcagatactggtatagagagacatctaggcgtagggggcaccagcgccacctttcacatggcagaagcctggacaatatggacataaggatttttgaatcagtcagggcaagtcgtgtgttttctattttgaatatatggaatatatttttgaatcacaggattttctactttaaagagtt